(19) United States
(12) Patent Application Publication
(10) Pub. No.: US 2018/0057883 A1
(43) Pub. Date: Mar. 1, 2018

(54) Compositions and Methods for the Diagnosis and Treatment of Lupus Nephritis

(71) Applicant: The Children's Hospital of Philadelphia, Philadelphia, PA (US)

(72) Inventors: Patricia Costa Reis, Lisbon (PT); Kathleen Sullivan, Philadelphia, PA (US)

(21) Appl. No.: 15/692,913

(22) Filed: Aug. 31, 2017

Related U.S. Application Data
(60) Provisional application No. 62/381,982, filed on Aug. 31, 2016.

(51) Int. Cl. C12Q 1/68; C07K 16/32; G01N 33/564

(52) U.S. Cl. CPC C12Q 1/6883; C07K 16/32; G01N 33/564; C07K 2317/76; A61K 2039/505; G01N 2800/104; C12Q 2600/158; C12Q 2600/178; G01N 2333/71

(57) ABSTRACT

Compositions and methods for diagnosing and treating lupus nephritis are disclosed.

### CROSS REFERENCE TO RELATED APPLICATION

This application claims priority to U.S. Provisional Application 62/381,982 filed Aug. 31, 2016, the entire disclosure being incorporated herein by reference as though set forth in full.

### FIELD OF THE INVENTION

This invention relates to the fields of autoimmune diseases and biomarkers therefore. More specifically, the invention provides specific biomarkers for identifying patients at risk for, or suffering from Lupus nephritis and Lupus nephritis flares.

### BACKGROUND OF THE INVENTION

Several publications and patent documents are cited throughout the specification in order to describe the state of the art to which this invention pertains. Each of these citations is incorporated herein by reference as though set forth in full.

Lupus nephritis (LN) is associated with considerable morbidity and mortality, particularly in children (1,2). The diagnosis of LN is based on renal biopsy findings, which have a modest predictive value for outcome. Lupus nephritis is histologically evident in most patients with systemic lupus erythematosus (SLE), even those without clinical manifestations of renal disease. Evaluating renal function in SLE patients is important because early detection and treatment of renal involvement can significantly improve renal outcome.

Patients with lupus nephritis may report symptoms which include fatigue, fever, rash, arthritis, serositis, or central nervous system [CNS] disease. These are more common with focal proliferative and diffuse proliferative lupus nephritis. In certain cases, asymptomatic lupus nephritis is present which is discovered during regular follow-up where laboratory abnormalities suggest active lupus nephritis. This form is more typical of mesangial or membranous lupus nephritis. In active nephritis, peripheral edema secondary to hypertension or hypoalbuminemia may be observed while extreme peripheral edema is more common with diffuse or membranous lupus nephritis. In patients suffering from diffuse lupus nephritis, headache, dizziness, visual disturbances, or signs of cardiac decompensation can be found.

Renal biopsy should be considered in any patient with SLE who has clinical or laboratory evidence of active nephritis, especially upon the first episode of nephritis.

Lupus nephritis is staged according to the classification revised by the International Society of Nephrology (ISN) and the Renal Pathology Society (RPS) in 2003, as follows:
Class I—Minimal mesangial lupus nephritis
Class II—Mesangial proliferative lupus nephritis
Class III—Focal lupus nephritis (active and chronic; proliferative and sclerosing)
Class IV—Diffuse lupus nephritis (active and chronic; proliferative and sclerosing;

segmental and global)
Class V—Membranous lupus nephritis
Class VI—Advanced sclerosis lupus nephritis

The principal goal of therapy in lupus nephritis is to normalize renal function or, at least, to prevent the progressive loss of renal function. Therapy differs, depending on the pathologic lesion. First, patients with clinical evidence of active, previously untreated lupus nephritis have a renal biopsy to classify the disease according to ISN/RPS criteria. Often, patients with lupus nephritis are treated with hydroxychloroquine, unless contraindicated. Glucocorticoids plus either cyclophosphamide intravenously or mycophenolate mofetil orally is frequently administered to patients with class III/IV disease while patients with class I/II nephritis do not require immunosuppressive therapy. Angiotensin-converting enzyme inhibitors or angiotensin-receptor blockers can be administered if proteinuria reaches or exceeds 0.5 g/day. Blood pressure should be maintained at or below 130/80 mm Hg. Patients with class V lupus nephritis are generally treated with prednisone for 1-3 months, followed by tapering for 1-2 years if a response occurs. If no response occurs, the drug is discontinued. Immunosuppressive drugs are generally not used unless renal function worsens or a proliferative component is present on renal biopsy samples.

Clearly, a need exists in the art for better approaches for diagnosis and management of lupus nephritis.

### SUMMARY OF THE INVENTION

In accordance with the present invention, a method of detecting HER2 in the urine of a patient at risk for, or having Lupus nephritis (LN) is provided. An exemplary method comprises the steps of contacting a urine sample obtained from said patient with an antibody having binding affinity for HER2 or a nucleic acid which hybridizes to a HER2 encoding nucleic acid and detecting formation of a specific binding pair comprising an antibody-HER2 immunocomplex or a duplex of a HER2 specific probe or primer and a HER2 encoding nucleic acid present in said urine sample, wherein the detection of said specific binding pair indicates an increased risk for, or the presence of LN. In some embodiments, the method entails detection of miR-26a and miR-30b levels in said sample. In other embodiments, VCAM1 and MCP-1 are also detected in said sample.

The diagnostic method can further comprise assessing the patient for additional LN symptoms selected from peripheral edema secondary to hypertension, headache, dizziness, visual disturbances, cardiac decompensation, hypoalbuminemia, fatigue, fever, rash, arthritis, serositis and central nervous system disease. The method can also comprise performing a renal biopsy on said patient to classify the type of LN present in said patient.

In certain aspects of the invention, the methods further comprise treating said subject with an agent effective alleviate symptoms of LN. Such agents include, without limitation, one or more of hydroxychloroquine, glucocorticoids, cyclophosphamide, mycophenolate mofetil, angiotensin converting enzyme inhibitors, angiotensin receptor blockers, prednisone, an immunosuppressive agent, hereceptin and trastuzumab.

HER2 protein in the sample can be detected by an immunological assay selected from the group consisting of enzyme linked immunosorbent assay (ELISA), Western blotting, immunoprecipitation assay, immunochromatography, radioimmunoassay (RIA), Radioimmunodiffusion, immunofluorescence assay (IFA), immunoblotting, Ouchterlony immunodiffusion, rocket immunoelectrophoresis, immunohistostaining, complement fixation assay, fluorescence activated cell sorting (FACS) and protein chip assay. The method can further comprise quantifying miR-26a and miR-30b in the urine sample via qRTPCR. In certain embodiments, VCAM1 and MCP-1 are also quantified in said sample.

In another embodiment of the invention, a kit for detecting lupus nephritis in a human using the methods described above is provided. An exemplary kit comprises antibodies immunologically specific for HER2, VCAM-1 and MCP-1; instructional materials for the use of said monoclonal antibody in detecting HER2, and optionally VCAM-1 and MCP-1 protein, and isolated HER2 protein for use as a control. The kits of the invention can further comprise a microtiter plate or a dipstick, said microtiter plate or dipstick having HER2 antibody or HER2 protein immobilized thereon. The kit can also further comprise reagents suitable for quantifying miR-26a and miR-30b using qRT-PCR. In yet another embodiment, the kit comprises probes or primers of sufficient complementarity to bind HER2 encoding nucleic acids, wherein the probes or primers are optionally being labeled with a non-naturally occurring detectable label.

### BRIEF DESCRIPTION OF THE DRAWINGS

FIGS. 1A-1C. miR-26a and miR-30b are decreased in the kidneys and urine of patients with LN. FIG. 1A. Levels of miR-26a, miR-30b and miR-4286 in the kidneys of pediatric LN patients (N=12) and controls (N=6) were measured by direct digital detection of molecular barcodes. FIG. 1B. miR-26a and miR-30b levels in the urine of adult LN patients (N=14) and controls (N=19) were analyzed by qRT-PCR. Error bars indicate the standard error of the mean. FIG. 1C. The principal component analysis of the miRNA expression in the kidneys of healthy controls (Normal) (N=6) and children with LN (LN) (N=17) and post-streptococcal glomerulonephritis (PS) (N=2) demonstrates distinct clusters.

FIGS. 2A-2D. Mesangial cells with miR-26a, miR-30b or miR-4286 KD have higher expression of genes related to cell cycle and proliferate more. FIG. 2A. Expression of different miRNA targets in mesangial cells with KD of miR-26a, miR-30b or miR-4286 and in vector-control cells was evaluated by qRT-PCR. FIG. 2B. MTT assays were performed to study cell proliferation in mesangial cells with KD of miR-26a, miR-30b or miR-4286 and in cells transduced with a lentivirus control vector and non-transfected cells. FIG. 2C. Mesangial cells with KD of miR-26a, miR-30b, or miR-4286 were cultured for six days and evaluated with Propidium iodide. FIG. 2D. MiRNA levels were measured by qRT-PCR after trastuzumab treatment (8 μg/ml) for 24 h. Error bars indicate the standard error of the mean. The experiments were performed at least three times.

FIGS. 3A-3D. HER2 expression was increased in the kidneys of LN patients (N=8) when compared to normal kidneys (N=2) and other proliferative glomerulonephritides (N=12). FIG. 3A. Positive controls were performed on HER2-positive breast cancer tissue. Negative controls were performed without the primary antibody. FIG. 3B. Kidneys from healthy donors. FIG. 3C. Kidneys from LN patients. FIG. 3D. Kidneys from patients with other proliferative glomerulonephritides: IgA Nephropathy (4), post-streptococcal glomerulonephritis (4) and granulomatosis with polyangiitis (4). Representative samples are shown in each case.

FIGS. 4A-4D. The number of HER2-positive cells detected per glomerulus in NZM2410 (N=9) mice was significantly higher than in Balb/c (N=3) or B6 mice (N=10). FIG. 4A. A negative control performed without the use of the primary antibody on NZM2410 mouse kidneys. FIG. 4B. HER2 immunohistochemistry on the kidneys of B6, Balb/c mice and NZM2410 mice. FIG. 4C. The number of HER2-positive cells per glomerulus in NZM2410, B6 and Balb/c mice. FIG. 4D. The number of HER2-positive cells in the glomeruli of NZM2410, according to proteinuria (mg/dL) and BUN at time of sacrifice (lower or higher than 30 mg/dL). Error bars indicate standard error of the mean.

FIGS. 5A-5D. α-interferon and IRF1 increase the expression of HER2 in human mesangial cells. FIG. 5A. IRF1 expression in cells transfected with IRF1 and with a vector-control, measured by immunofluorescence and qRT-PCR (CTCF—corrected total cell fluorescence), demonstrate successful overexpression. FIG. 5B. Expression of HER2 in mesangial cells transfected with IRF1 or the control vector was studied by qRT-PCR. FIG. 5C. MiR-26a and miR-30b levels were measured in cells transfected with IRF1 and vector-control by qRT-PCR. FIG. 5D. Expression of HER2 in mesangial cells, with and without previous α-interferon exposure, was measured by qRT-PCR. Error bars indicate the standard error of the mean. The experiments were performed at least three times.

FIGS. 6A-6D. HER2 is increased in the urine of patients with active LN and it is associated with disease activity. FIG. 6A. HER2 levels (pg/mL) were measured by ELISA in the urine of adult patients with active LN (N=47) and in sex and age-matched healthy controls (N=26). FIG. 6B. HER2 levels (pg/mg Cr) were analyzed in 14 patients from two different time-points: non-active LN and a LN flare. FIG. 6C. Urinary HER2 levels (pg/mg Cr) were analyzed according to different histologic classes of LN (N=19). FIG. 6D. HER2 levels (pg/mg Cr) were expressed according to the urine protein:creatinine ratio (mg/mg) (N=46), MCP-1 (pg/mg Cr) (N=31) and VCAM-1 levels (ng/mg Cr) (N=31). Error bars indicate the standard error of the mean.

### DETAILED DESCRIPTION OF THE INVENTION

MiRNAs in the kidneys of lupus nephritis patients and controls were analyzed by molecular digital detection. Urinary miRNAs were measured by qRT-PCR. Target gene expression in human mesangial cells was evaluated by arrays and qRT-PCR. HER2 was analyzed by immunohistochemistry in kidneys of LN patients and in a lupus murine model. Urinary HER2, MCP-1 and VCAM-1 were measured by ELISA.

Decreased miR-26a, miR-30b and miR-4286 levels were found in LN, a set of miRNAs that regulate cell proliferation. HER2, a protein that regulates these miRNAs in breast cancer cells, was overexpressed in lupus-prone NZM2410 mice and in human LN kidneys, and was found to be upregulated by a type I interferon. Urinary HER2 was increased in LN, reflected disease activity, and levels correlated with two other recognized LN biomarkers, MCP-1 and VCAM-1.

The present inventors have discovered that the kidney miRNA pattern is broadly changed in LN, which contributes to uncontrolled cell proliferation and disease symptoms. MiR-26a and miR-30b are decreased in the kidneys and urine of LN patients, and directly regulate the cell cycle. The levels of these miRNAs are controlled by HER2, which is overexpressed in NZM2410 mice and in the kidneys and urine of LN patients. MiR-26a, miR-30b and HER2 are thus new LN biomarkers and blocking HER2 provides a new strategy to decrease cell proliferation and damage in this disease.

### I. Definitions

The definitions below are provided to facilitate the understanding of the invention. The receptor tyrosine-protein kinase erbB-2, (also known as CD340 (cluster of differentiation 340), proto-oncogene Neu, Erbb2 (rodent), or ERBB2 (human), is a protein that in humans is encoded by the ERBB2 gene, which is also frequently called HER2 (from human epidermal growth factor receptor 2) or HER2/neu. “HER2” is a member of the human epidermal growth factor receptor (HER/EGFR/ERBB) family. Amplification or over-expression of this oncogene has been shown to play an important role in the development and progression of certain aggressive types of breast cancer.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. As used herein, the term refers to a molecule comprising at least complementarity-determining region (CDR) 1, CDR2, and CDR3 of a heavy chain and at least CDR1, CDR2, and CDR3 of a light chain, wherein the molecule is capable of binding to antigen. The term antibody includes, but is not limited to, fragments that are capable of binding antigen, such as Fv, single-chain Fv (scFv), Fab, Fab′, and (Fab′)₂. The term antibody also includes, but is not limited to, chimeric antibodies, humanized antibodies, human antibodies, and antibodies of various species such as mouse, cynomolgus monkey, etc. The term “heavy chain” refers to a polypeptide comprising at least a heavy chain variable region, with or without a leader sequence. In some embodiments, a heavy chain comprises at least a portion of a heavy chain constant region. The term “full-length heavy chain” refers to a polypeptide comprising a heavy chain variable region and a heavy chain constant region, with or without a leader sequence.

The term “heavy chain variable region” refers to a region comprising a heavy chain complementary determining region (CDR) 1, framework region (FR) 2, CDR2, FR3, and CDR3 of the heavy chain

The term “light chain” refers to a polypeptide comprising at least a light chain variable region, with or without a leader sequence. In some embodiments, a light chain comprises at least a portion of a light chain constant region. The term “full-length light chain” refers to a polypeptide comprising a light chain variable region and a light chain constant region, with or without a leader sequence. The term “light chain variable region” refers to a region comprising a light chain CDR1, FR2, HVR2, FR3, and HVR3. In some embodiments, a light chain variable region also comprises an FR1 and/or an FR4. See, e.g., Kabat Sequences of Proteins of Immunological Interest (1987 and 1991, NIH, Bethesda, Md.).

A “chimeric antibody” refers to an antibody in which a portion of the heavy and/or light chain is derived from a particular source or species, while the remainder of the heavy and/or light chain is derived from a different source or species. In some embodiments, a chimeric antibody refers to an antibody comprising at least one variable region from a first species (such as mouse, rat, cynomolgus monkey, etc.) and at least one constant region from a second species (such as human, cynomolgus monkey, etc.). In some embodiments, a chimeric antibody comprises at least one mouse variable region and at least one human constant region. In some embodiments, a chimeric antibody comprises at least one cynomolgus variable region and at least one human constant region. In some embodiments, all of the variable regions of a chimeric antibody are from a first species and all of the constant regions of the chimeric antibody are from a second species.

A “humanized antibody” refers to an antibody in which at least one amino acid in a framework region of a non-human variable region has been replaced with the corresponding amino acid from a human variable region. In some embodiments, a humanized antibody comprises at least one human constant region or fragment thereof. In some embodiments, a humanized antibody is an Fab, an scFv, a (Fab′)₂, etc.

A “human antibody” as used herein refers to antibodies produced in humans, antibodies produced in non-human animals that comprise human immunoglobulin genes, such as XenoMouse®, and antibodies selected using in vitro methods, such as phage display, wherein the antibody repertoire is based on a human immunoglobulin sequences.

The terms “inhibition” or “inhibit” refer to a decrease or cessation of any event (such as protein ligand binding) or to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. It is not necessary that the inhibition or reduction be complete. For example, in certain embodiments, by “reduce” or “inhibit” is meant the ability to cause an overall decrease of 20% or greater. In another embodiment, by “reduce” or “inhibit” is meant the ability to cause an overall decrease of 50% or greater. In yet another embodiment, by “reduce” or “inhibit” is meant the ability to cause an overall decrease of 75%, 85%, 90%, 95%, or greater.

The term “solid matrix” as used herein refers to any format, such as beads, microparticles, a microarray, the surface of a microtitration well or a test tube, a dipstick or a filter. The material of the matrix may be polystyrene, cellulose, latex, nitrocellulose, nylon, polyacrylamide, dextran or agarose.

The phrase “consisting essentially of” when referring to a particular nucleotide or amino acid means a sequence having the properties of a given SEQ ID NO: or compound. For example, when used in reference to an amino acid sequence, the phrase includes the sequence per se and molecular modifications that would not affect the functional and novel characteristics of the sequence. Similarly, the phrase refers to compounds with modifications that do not affect the functional and novel characteristics of the parent compound. Methods can also consist essentially of a recited series of steps.

With regard to nucleic acids used in the invention, the term “isolated nucleic acid” is sometimes employed. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a prokaryote or eukaryote. An “isolated nucleic acid molecule” may also comprise a cDNA molecule. An isolated nucleic acid molecule inserted into a vector is also sometimes referred to herein as a recombinant nucleic acid molecule.

With respect to RNA molecules, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form. MicroRNAs (miRNAs) are small, RNA molecules encoded in the genomes of plants and animals. These highly conserved, ˜21-mer RNAs regulate the expression of genes by binding to the 3′-untranslated regions (3′-UTR) of specific mRNAs.

By the use of the term “enriched” in reference to nucleic acid it is meant that the specific DNA or RNA sequence constitutes a significantly higher fraction (2-5 fold) of the total DNA or RNA present in the cells or solution of interest than in normal cells or in the cells from which the sequence was taken. This could be caused by a person by preferential reduction in the amount of other DNA or RNA present, or by a preferential increase in the amount of the specific DNA or RNA sequence, or by a combination of the two. However, it should be noted that “enriched” does not imply that there are no other DNA or RNA sequences present, just that the relative amount of the sequence of interest has been significantly increased.

It is also advantageous for some purposes that a nucleotide sequence be in purified form. The term “purified” in reference to nucleic acid does not require absolute purity (such as a homogeneous preparation); instead, it represents an indication that the sequence is relatively purer than in the natural environment (compared to the natural level, this level should be at least 2-5 fold greater, e.g., in terms of mg/ml). Individual clones isolated from a cDNA library may be purified to electrophoretic homogeneity. The claimed DNA molecules obtained from these clones can be obtained directly from total DNA or from total RNA. The cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process includes the construction of a cDNA library from mRNA and isolation of distinct cDNA clones and yields an approximately 10⁻⁶-fold purification of the native message. Thus, purification of at least one order of magnitude, preferably two or three orders, and more preferably four or five orders of magnitude is expressly contemplated. Thus the term “substantially pure” refers to a preparation comprising at least 50-60% by weight the compound of interest (e.g., nucleic acid, oligonucleotide, etc.). More preferably, the preparation comprises at least 75% by weight, and most preferably 90-99% by weight, the compound of interest. Purity is measured by methods appropriate for the compound of interest.

The term “complementary” describes two nucleotides that can form multiple favorable interactions with one another. For example, adenine is complementary to thymine as they can form two hydrogen bonds. Similarly, guanine and cytosine are complementary since they can form three hydrogen bonds. Thus if a nucleic acid sequence contains the following sequence of bases, thymine, adenine, guanine and cytosine, a “complement” of this nucleic acid molecule would be a molecule containing adenine in the place of thymine, thymine in the place of adenine, cytosine in the place of guanine, and guanine in the place of cytosine. Because the complement can contain a nucleic acid sequence that forms optimal interactions with the parent nucleic acid molecule, such a complement can bind with high affinity to its parent molecule. Levels of complementarity between selectively hybridizing nucleic acids can vary but is typically greater than 80% and is preferably between 90-95%.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”) with enough sequence specificity to distinguish the target sequence over non target sequences. In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence. For example, specific hybridization can refer to a sequence that hybridizes to any lupus nephritis specific marker nucleic acid, but does not hybridize to other nucleotides. Also polynucleotide that “specifically hybridizes” may hybridize only to a single LN-specific marker herein. Appropriate conditions enabling specific hybridization of single stranded nucleic acid molecules of varying complementarity are well known to those of skill in the art.

For instance, one common formula for calculating the stringency conditions required to achieve hybridization between nucleic acid molecules of a specified sequence homology is set forth below (Sambrook et al., Molecular Cloning, Cold Spring Harbor Laboratory (1989):

Tₘ=81.5″C+16.6 Log [Na+]+0.41(% G+C)−0.63(% formamide)−600/#bp in duplex

As an illustration of the above formula, using [Na+]=[0.368] and 50% formamide, with GC content of 42% and an average probe size of 200 bases, the Tₘ is 57″C. The Tₘ of a DNA duplex decreases by 1-1.5″C with every 1% decrease in homology. Thus, targets with greater than about 75% sequence identity would be observed using a hybridization temperature of 42″C. The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5×Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5×Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C., and washed in 0.1×SSC and 0.5% SDS at 65° C. for 15 minutes.

The term “oligonucleotide,” as used herein is defined as a nucleic acid molecule comprised of two or more ribo- or deoxyribonucleotides, preferably more than three. The exact size of the oligonucleotide will depend on various factors and on the particular application and use of the oligonucleotide. Oligonucleotides, which include probes and primers, can be any length from 3 nucleotides to the full length of the nucleic acid molecule, and explicitly include every possible number of contiguous nucleic acids from 3 through the full length of the polynucleotide. Preferably, oligonucleotides are at least about 10 nucleotides in length, more preferably at least 15 nucleotides in length, more preferably at least about 20 nucleotides in length.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains between 10-25, 15-50 and 15 to 100 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25, 15-35, 10-40 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

A “specific binding pair” comprises a specific binding member (sbm) and a binding partner (bp) that have a particular specificity for each other and which in normal conditions bind to each other in preference to other molecules. Examples of specific binding pairs are antigens and antibodies, ligands and receptors and complementary nucleotide sequences. The skilled person is aware of many other examples. Further, the term “specific binding pair” is also applicable where either or both of the specific binding member and the binding partner comprise a part of a large molecule. In embodiments in which the specific binding pair comprises nucleic acid sequences, they will be of a length to hybridize to each other under conditions of the assay, preferably greater than 10 nucleotides long, more preferably greater than 15 or 20 nucleotides long. Typically, one or both members of a specific binding pair will comprise a non naturally occurring detectable label.

“Sample” or “patient sample” or “biological sample” generally refers to a sample which may be tested for a particular molecule, preferably a LN specific marker molecule, such as an miRNA and/or HER2 polypeptide as described herein. Samples may include but are not limited to cells, body fluids, including blood, serum, plasma, urine, saliva, tears, pleural fluid and the like. In a preferred embodiment, the sample is a urine sample.

The terms “agent” and “test compound” are used interchangeably herein and denote a chemical compound, a mixture of chemical compounds, a biological macromolecule, or an extract made from biological materials such as bacteria, plants, fungi, or animal (particularly mammalian) cells or tissues. Biological macromolecules include siRNA, shRNA, antisense oligonucleotides, peptides, peptide/DNA complexes, and any nucleic acid based molecule which exhibits the capacity to modulate the activity of the SNP containing nucleic acids described herein or their encoded proteins. Agents are evaluated for potential biological activity by inclusion in screening assays described hereinbelow.

“Treatment,” as used herein, covers any administration or application of a therapeutic for a disease (also referred to herein as a “disorder” or a “condition”) in a mammal, including a human, and includes inhibiting the disease or progression of the disease, inhibiting or slowing the disease or its progression, arresting its development, partially or fully relieving the disease, partially or fully relieving one or more symptoms of a disease, or restoring or repairing a lost, missing, or defective function; or stimulating an inefficient process.

The term “effective amount” or “therapeutically effective amount” refers to an amount of a drug effective for treatment of a disease or disorder in a subject, such as to partially or fully relieve one or more symptoms. In some embodiments, an effective amount refers to an amount effective, at dosages and for periods of time necessary, to achieve the desired therapeutic or prophylactic result.

### II. Methods for Detection of HER2 and miRNAs Indicative of LN

### ELISA Assay

In the present invention, an enzyme-linked immunosorbent assay (ELISA) assay may be used to detect and quantitate HER2 in a urine sample. For example, antibodies to HER2 may be immobilized onto a selected surface, for example, a surface such as a microtiter well, a membrane, a filter, a bead or a dipstick. After washing to remove incompletely adsorbed material, it is desirable to bind or coat the surface with a non-specific agent that is known to be antigenically neutral with regard to the test sample, e.g., bovine serum albumin (BSA), casein or solutions of powdered milk. This allows for blocking of non-specific adsorption sites on the immobilizing surface and thus reduces the background caused by non-specific binding of antibody to antigen on the surface.

Detection of HER2 protein in urine may be accomplished by other techniques known in the art, e.g., Western blots, 2D gel electrophoresis and the like.

As appreciated by one skilled in the art, an enzyme-linked immunosorbent assay (ELISA) may be employed to detect proteins in urine, specifically HER2 protein in urine. In one aspect, the present invention provides an ELISA in detecting HER2 protein in human urine. The presence of HER2 protein is an indication of lupus nephritis.

In one embodiment, the present invention provides an initial step of an ELISA in which an anti-HER2 antibody is immobilized onto a surface (for example by passive adsorption known as coating). For purposes of this application, HER2 and detectable fragments thereof may be detected. Anti-HER2 antibody may recognize full-length HER2 protein as well as fragments thereof via its recognition of specific epitopes. Immobilization of anti-HER2 antibody may be performed on any inert support (or solid support) that is useful in immunological assays. Examples of commonly used inert supports include small sheets, Sephadex and assay plates manufactured from polyethylene, polypropylene or polystyrene. In a preferred embodiment, the immobilized anti-HER2 antibody is coated on a microtiter plate that allows analysis of several samples at one time. More preferably, the microtiter plate is a microtest 96-well ELISA plate, such as those sold under the name Nunc Maxisorb or Immulon. Anti-HER2 antibody can also be immobilized on a dip stick to facilitate testing of urine.

Antibody immobilization is often conducted in the presence of a buffer at an optimum time and temperature optimized by one skilled in the art. Suitable buffers should enhance immobilization without affecting the antigen binding properties. Sodium carbonate buffer (e.g., 50 mM, pH 9.6) is a representative suitable buffer, but others such as Tris-HCl buffer (20 mM, pH 8.5), phosphate-buffered saline (PBS) (10 mM, pH 7.2-7.4) are also used. Optimal coating buffer pH will be dependent on the antigen(s) being immobilized. Optimal results may be obtained when a buffer with pH value 1-2 units higher than the isoelectric point (pI) value of the protein is used. Incubation time ranges from 2-8 hours to overnight. Incubation may be performed at temperatures ranging from 4-37° C. Preferably, immobilization takes place overnight at 4° C. The plates may be stacked and coated long in advance of the assay itself, and then the assay can be carried out simultaneously on several samples in a manual, semi-automatic, or automatic fashion, such as by using robotics.

Blocking agents are used to eliminate non-specific binding sites in order to prevent unwanted non-specific antibody binding to the plate. Examples of appropriate blocking agents include detergents (for example, Tween-20, Tween-80, Triton-X 100, sodium dodecyl sulfate), gelatin, bovine serum albumin (BSA), egg albumin, casein, non-fat dried milk and the like. Preferably, the blocking agent is BSA. Concentrations of blocking agent may easily be optimized (e.g. BSA at 1-5%). The blocking treatment typically takes place under conditions of ambient temperatures for about 1-4 hours, preferably 1.5 to 3 hours.

After coating and blocking, urine from control subjects or patients suspected of LN can be added to the immobilized antigens in the plate. Concentrated urine suspended in Phosphate Buffered Saline (PBS) containing 0.5% BSA, 0.05% TWEEN 20® detergent may be used. TWEEN 20® acts as a detergent to reduce non-specific binding.

The conditions for incubation of the biological sample and immobilized antigen are selected to maximize sensitivity of the assay and to minimize dissociation. Preferably, the incubation is accomplished at a constant temperature, ranging from about 0° C. to about 40° C., preferably from about 22 to 25° C. to obtain a less variable, lower coefficient of variant (CV) than at, for example, room temperature. The time for incubation depends primarily on the temperature, being generally no greater than about 10 hours to avoid an insensitive assay. Preferably, the incubation time is from about 0.5 to 3 hours, and more preferably 1.5-3 hours at room temperature to maximize binding to immobilized capture antigen.

Following incubation of the biological sample (urine) and immobilized anti-HER2 antibody, unbound biological sample is separated from the immobilized antibody by washing. The solution used for washing is generally a buffer (“washing buffer”) with a pH determined using the considerations and buffers described above for the incubation step, with a preferable pH range of about 6-9. Preferably, pH is 7. The washing may be done three or more times. The temperature of washing is generally from refrigerator to moderate temperatures, with a constant temperature maintained during the assay period, typically from about 0-40° C., more preferably about 4-30° C. For example, the wash buffer can be placed in ice at 4° C. in a reservoir before the washing, and a plate washer can be utilized for this step.

Next, the immobilized capture anti-HER2 antibody and biological sample (i.e., urine) are contacted with a detectable antibody at a time and temperature optimized by one skilled in the art. Detectable antibody may include a monoclonal antibody or a polyclonal antibody. These antibodies may be directly or indirectly conjugated to a label. Suitable labels include moieties that may be detected directly, such as fluorochrome, radioactive labels, and enzymes, that must be reacted or derivatized to be detected. Examples of such labels include the radioisotopes 32P, 14C, 125I, 3H, and 131I, fluorophores such as rare earth chelates or fluorescein and its derivatives, rhodamine and its derivatives, horseradish peroxidase (HRP), alkaline phosphatase, and the like. Preferably, the detection antibody is a goat anti-human IgG polyclonal antibody that binds to human IgG and is directly conjugated to HRP. Incubation time ranges from 30 minutes to overnight, preferably about 60 minutes. Incubation temperature ranges from about 20-40° C., preferably about 22-25° C., with the temperature and time for contacting the two being dependent on the detection means employed.

The conjugation of such labels to the antibody, including the enzymes, is a standard manipulative procedure for one of ordinary skill in immunoassay techniques. See, for example, O'Sullivan et al. “Methods for the Preparation of Enzyme-antibody Conjugates for Use in Enzyme Immunoassay,” in Methods in Enzymology, ed. J. J. Langone and H. Van Vunakis, Vol. 73 (Academic Press, New York, N.Y., 1981), pp. 147-166.

In one embodiment, after the complex formation between HER2 protein and anti-HER2 antibody, the antibody binding to antigen (i.e., HER2 protein) is assessed by detecting a label on the primary antibody. In another embodiment, the primary antibody is assessed by detecting binding of a secondary antibody or reagent to the primary antibody. In a further embodiment, the secondary antibody is labeled. Many means are known in the art for detecting binding in an immunoassay and are within the scope of the present invention. For example, to select specific epitopes of recombinant or synthetic polypeptide, one may assay antibody binding in an ELISA assay wherein the polypeptides or its fragments containing such epitope.

In another aspect, the present invention provides direct immobilizing urine samples (containing HER2 protein) onto a solid support (e.g., microtiter plates or dipstick). This is also known as antigen-down ELISA (i.e., indirect ELISA). In such an assay, a microtiter plate is coated with a sample containing a certain antigen (e.g., HER2 protein). After allowing for adsorption of the antigen onto the plate, and washing off all non-bound materials, an antibody is added to the plate and the excess washed off. Prior to addition of the antibody, one skilled in the art would appreciate blocking non-specific bindings with appropriate blocking agents (e.g., Tween-20, Tween-80, Triton-X 100, sodium dodecyl sulfate), gelatin, bovine serum albumin (BSA), egg albumin, casein, non-fat dried milk and the like). The added antibody may have already been labeled with a reporter molecule to permit the generation of a signal to be read by known techniques (e.g., microtiter plate reader).

An anti-HER2 protein is directly added to allow the formation of HER2 and anti-HER2 antibody complex. Optimal conditions for antigen-antibody complex formation may be conveniently adjusted by one of ordinary skilled in the art. Unbound antibody is removed by washing; generally by a buffer. Detectable antibody may include a monoclonal antibody or a polyclonal antibody. These antibodies may be directly or indirectly conjugated to a label (as described above).

Alternatively, an enzyme conjugated secondary antibody can be used for detection of antigen-antibody complex. Upon addition of a suitable chromogen substrate, a color develops which is used as an indicator of the amount of antigen present.

IIa. Dipstick Technology

U.S. Pat. No. 4,366,241, and Zuk, EP-A 0 143 574 describe migration type assays in which a membrane is impregnated with the reagents needed to perform the assay. An analyte detection zone is provided in which labeled analyte is bound and assay indicia is read.

U.S. Pat. No. 4,770,853, WO 88/08534, and EP-A 0 299 428 describe migration assay devices which incorporate within them reagents which have been attached to colored direct labels, thereby permitting visible detection of the assay results without addition of further substances.

U.S. Pat. No. 4,632,901, disclose a flow-through type immunoassay device comprising antibody (specific to a target antigen analyte) bound to a porous membrane or filter to which is added a liquid sample. As the liquid flows through the membrane, target analyte binds to the antibody. The addition of sample is followed by addition of labeled antibody. The visual detection of labeled antibody provides an indication of the presence of target antigen analyte in the sample.

EP-A 0 125 118, disclose a sandwich type dipstick immunoassay in which immunochemical components such as antibodies are bound to a solid phase. The assay device is “dipped” for incubation into a sample suspected of containing unknown antigen analyte. Enzyme-labeled antibody is then added, either simultaneously or after an incubation period. The device next is washed and then inserted into a second solution containing a substrate for the enzyme. The enzyme-label, if present, interacts with the substrate, causing the formation of colored products which either deposit as a precipitate onto the solid phase or produce a visible color change in the substrate solution.

EP-A 0 282 192, disclose a dipstick device for use in competition type assays.

U.S. Pat. No. 4,313,734 describes the use of gold sol particles as a direct label in a dipstick device.

U.S. Pat. No. 4,786,589 describes a dipstick immunoassay device in which the antibodies have been labeled with formazan.

U.S. Pat. No. 5,656,448 pertains to dipstick immunoassay devices comprising a base member and a single, combined sample contact zone and test zone, wherein the test zone incorporates the use of symbols to detect analytes in a sample of biological fluid. A first immunological component, an anti-immunoglobulin capable of binding to an enzyme-labeled antibody, is immobilized in a control indicator portion. A second immunological component, capable of specifically binding to a target analyte which is bound to the enzyme-labeled antibody to form a sandwich complex, is immobilized in a test indicia portion. The enzyme-labeled antibody produces a visual color differential between a control indicia portion and a non-indicia portion in the test zone upon contact with a substrate. The device additionally includes a first polyol and a color differential enhancing component selected from the group consisting of an inhibitor to the enzyme and a competitive secondary substrate for the enzyme distributed throughout the non-indicia portion of the test zone.

Detection of miRNAs

The sequences of the miRNAs associated with LN are found in the publically available miRNA data base. Methods for detecting specific miRNA are well known to the skilled artisan and include without limitation quantitative reverse transcription polymerase chain reaction (qRT-PCR) as described herein below. Other approaches include the use of microarrays having miRNA probes affixed thereto and reagents suitable for detecting hybridization of the probes to miRNAs in the sample if present.

### Detection of HER2 Encoding Nucleic Acids.

In certain embodiments, urine samples can be assessed for HER2 encoding nucleic acids in order to diagnose an increased risk for Lupus nephritis. Methods for detecting HER2 encoding nucleic acids are known to the skilled artisan, and include without limitation, polymerase chain reaction, northern and southern blotting assays, and quantitative reverse transcription polymerase chain reaction (qRT-PCR) as described herein below. Other approaches include the use of microarrays having HER2 specific probes affixed thereto and reagents suitable for detecting hybridization of the probes to HER2 encoding nucleic acids in the sample if present.

### III. Kits

In still further embodiments, the present invention concerns immunodetection kits for use with the immunodetection methods described above. The kits will include antibodies to HER2., and may contain other reagents as well. The immunodetection kits will thus comprise, in suitable container means, a first antibody that binds to HER2, and optionally a second and distinct antibody to HER2.

In certain embodiments, the antibody to HER2 may be pre-bound to a solid support, such as a column matrix, a microtitre plate, a filter, a membrane, a bead or a dipstick. The immunodetection reagents of the kit may take any one of a variety of forms, including antibodies to HER2 containing detectable labels. As noted above, a number of exemplary labels are known in the art and all such labels may be employed in connection with the present invention. Antibodies to monocyte chemotactic protein 1 (MCP1) and/or vascular cell adhesion molecule 1 (VCAM-1) may also be included in the kit and optionally immobilized to the same or a different solid support.

The kits may further comprise a suitably aliquoted composition of HER2, whether labeled or unlabeled, as may be used to prepare a standard curve for a detection assay. The components of the kits may be packaged either in aqueous media or in lyophilized form.

The kits may also contain probes or primers suitable for detection of HER2 encoding nucleic acids, miR-26a and MiR30b. Such primers or probes can optionally comprise a non-naturally occurring detectable label and reagents suitable for detecting specific binding between said HER2 encoding nucleic acids and/or miRNA if present in the sample and the probe or primers specific for these molecules.

The container means of the kits will generally include at least one vial, test tube, flask, bottle, syringe or other container means, into which the antibody may be placed, or preferably, suitably aliquoted. The kits of the present invention will also typically include a means for containing the antibody, antigen, and any other reagent containers in close confinement for commercial sale. Such containers may include injection or blow-molded plastic containers into which the desired vials are retained.

### IV. Treatments

The treatment of lupus nephritis can vary depending on the severity of the disease as described above. Given that HER2 overexpression has now been associated with LN symptoms, herceptin may now be employed as an effective treatment for this autoimmune disorder alone or in combination with other pharmaceutical compounds currently employed to alleviate LN symptoms.

The following materials and methods are provided to facilitate the practice of the present invention.

### Patients and Kidney Specimens

Paraffin-embedded kidney samples were selected from pediatric patients with LN or post-streptococcal glomerulonephritis and controls. The controls were normal kidneys from adult donors and one sample of normal kidney tissue from a child with nephroblastoma. Table 1 summarizes the demographic and clinical characteristics of the LN cohort. A pathologist confirmed the diagnoses and classified the LN findings according to the International Society of Nephrology and the Renal Pathology Society criteria (13). The institutional review board (IRB) of The Children's Hospital of Philadelphia approved the study.

TABLE 1
Demographic and clinical Information on samples used to study
miRNAs in the kidneys

| | Number (%) or |
|---|---|
| Characteristics | Mean ± S.D. |
| Gender | |
| Female | 8 (67%) |
| Male | 4 (33%) |
| Ethnicity | |
| African-American | 5 (42%) |
| Asian | 2 (17%) |
| Caucasian | 3 (25%) |
| Hispanic | 2 (17%) |
| Age at SLE diagnosis (years) | 13 ± 2 |
| Age at LN diagnosis (years) | 14 ± 3 |
| Age at time of kidney biopsy (years) | 14 ± 3 |
| Biopsy during the first year after LN diagnosis | 11 (92%) |
| SLE Manifestations until the time of kidney biopsy | |
| Malar rash | 4 (33%) |
| Oral ulcers | 2 (17%) |
| Non-erosive arthritis | 7 (58%) |
| Pleuritis or Pericarditis | 1 (8%) |
| Renal disorder | 12 (100%) |
| Neurological disorder | 1 (8%) |
| Hematological disorder | 8 (67%) |
| Immunologic disorder | 10 (83%) |
| Positive anti-nuclear antibody | 12 (100%) |
| Antibody Profile | |
| Anti-DNA | 9 (75%) |
| Anti-Sm | 3 (25%) |
| Anti-RNP | 4 (33%) |
| Anti-SSA | 2 (17%) |
| Anti-SSB | 1 (8%) |
| Anti-phospholipids antibodies | 2 (17%) |
| SELENA-SLEDAI at time of Kidney biopsy | 17 ± 7 |
| Renal SELENA-SLEDAI at time of Kidney Biopsy | 11 ± 4 |
| Urinary casts | 9 (75%) |
| Hematuria | 8 (67%) |
| Proteinuria | 10 (83%) |
| Pyuria | 3 (25%) |
| LN active at time of kidney biopsy | 12 (100%) |
| Treatment at time of kidney biopsy | |
| No treatment | 8 (67%) |
| Hydroxychloroquine | 2 (17%) |
| Prednisone | 4 (33%) |
| Mycophenolate mofetil | 1 (8%) |
| Azathioprine | 0 (0%) |
| Cyclophosphamide | 3 (25%) |
| Rituximab | 0 (0%) |
| LN Class | |
| LN class III | 4 (33%) |
| LN class IV | 8 (67%) |

RNA Extraction and miRNA Quantification

RNA was extracted with the FFPE miRNeasy kit (Qiagen) and analyzed using the Nanodrop 2000. High purity samples were chosen, according to the absorbance ratios 260/280 and 260/230. Seven hundred and thirty four miRNAs were analyzed by direct digital detection of molecular barcodes, with the nCounter assay (NanoString). This method is ideal for fragmented RNA samples and is highly sensitive for miRNAs. Six negative miRNA assay controls, six positive miRNA spikes, and five housekeeping mRNAs controls (ACTB, B2M, GAPDH, RPL19 and RPL0) were also quantified. All samples were normalized within recommended guidelines.

The data were normalized to the sum of six positive control miRNA spikes. To account for differences in miRNA content in each sample, the data was normalized to the sum of all miRNA counts for each assay. Principal component analysis was performed using the R statistical computing language and pathway analysis was done with Ingenuity. Statistical significance was defined as the Benjamini-Hochberg false discovery rate <0.05.

The miR-26a and miR-30b levels were studied in the same cohort by quantitative real-time polymerase chain reaction (qRT-PCR), using Taqman miRNA assays (Applied Biosystems) and the 7900HT. Relative quantification was applied using spiked Caenorhabditis elegans miRNA-238 as control (Qiagen). Commercially available primers were purchased from Applied Biosystems (Table 2).

TABLE 2
Primers for qRT-PCR and Lentiviruses used

| | | | |
|---|---|---|---|
| Name | Company | Catalog Number | Code |

Primers for miRNA quantification

| | | | |
|---|---|---|---|
| hsa-miR-26a | Applied Biosystems | 4427975 | 000405 |
| hsa-miR-30b | Applied Biosystems | 4427975 | 000602 |
| cel-miR-39 | Applied Biosystems | 4427975 | 000200 |
| cel-miR-238 | Applied Biosystems | 4427975 | 000248 |

Primers for mRNA quantification

| CCNE2 | Applied Biosystems | 4448892 | Hs00180319_m1 |
|---|---|---|---|
| FADD | Applied Biosystems | 4448892 | Hs04187499_m1 |
| E2F8 | Applied Biosystems | 4448892 | Hs01079645_g1 |
| IL32 | Applied Biosystems | 4448892 | Hs00992441_m1 |
| IL7R | Applied Biosystems | 4448892 | Hs00902338_g1 |
| MAD2L1 | Applied Biosystems | 4448892 | Hs00277143_m1 |
| MYBL1 | Applied Biosystems | 4448892 | Hs00277143_m1 |
| PARP2 | Applied Biosystems | 4448892 | Hs01003785_g1 |
| POLQ | Applied Biosystems | 4448892 | Hs00981375_m1 |
| PTGR2 | Applied Biosystems | 4448892 | Hs01584044_m1 |
| HER2 | Applied Biosystems | 4448892 | Hs01001580_m1 |
| IRF1 | Applied Biosystems | 4448892 | Hs00971960_m1 |
| Name | Company | Code | Description |

Lentiviruses used for the knock-downs of the miRNAs of interest

| | | | |
|---|---|---|---|
| hsa-miR-26a | Abm | mh35378 | LentimiRa-Off- |
| | | | hsa-miR26a |
| hsa-miR-30b | Abm | mh35422 | LentimiRa-Off- |
| | | | hsa-miR30b |
| hsa-miR-4286 | Abm | mh35785 | LentimiRa-Off- |
| | | | hsa-miR4286 |
| Control lentivirus | Abm | m008 | Lenti-III-miR off |
| | | | Control Virus |

Urine miRNA Detection

Urine samples were collected from healthy individuals and adult patients followed at the Hospital of the University of Pennsylvania. All the patients had at least one encounter with clinical and laboratory evidence of active LN in the past three years (hematuria, proteinuria, pyuria and/or urinary casts). Table 3 displays the characteristics of this cohort. the urine samples were centrifuged at 3000 rpm at 4° C. for 30 min and aliquots of 100 μL of the supernatants with 500 μL of QIAzol lysis reagent were kept at −80° C. RNA was extracted from these samples using Qiagen miRNeasy serum/plasma kit.

TABLE 3
Demographic and clinical information regarding the cohort
used to study miRNAs in the urine *

| | Number (%) or |
|---|---|
| Characteristics | Mean ± S.D. |
| Gender | |
| Female | 12 (86%) |
| Male | 2 (14%) |
| Ethnicity | |
| African-American | 8 (57%) |
| Caucasian | 4 (29%) |
| Asian | 1 (7%) |
| Hispanic | 1 (7%) |
| Age at SLE diagnosis (years) | 20 ± 11 |
| Age at LN diagnosis (years) | 23 ± 13 |
| Age at time of urine collection (years) | 37 ± 10 |
| Time between LN diagnosis and urine collection | 14 ± 10 |
| (years) | |
| Clinical manifestations of SLE | |
| Malar Rash | 8 (57%) |
| Discoid Rash | 5 (36%) |
| Photosensitivity | 3 (21%) |
| Oral ulcers | 4 (29%) |
| Arthritis | 11 (79%) |
| Serositis | 2 (14%) |
| Renal Disorder | 14 (100%) |
| Neurologic Disorder | 5 (36%) |
| Hematologic Disorder | 6 (43%) |
| Immunologic Disorder | 11 (86%) |
| Positive anti-nuclear antibody | 14 (100%) |
| Antibody Profile | |
| Anti-DNA | 9 (71%) |
| Anti-Sm | 3 (21%) |
| Anti-RNP | 5 (36%) |
| Anti-SSA | 4 (29%) |
| Anti-SSB | 0 (0%) |
| Anti-phospholipids antibodies | 1 (7%) |
| Kidney Biopsy | |
| LN Class IV | 10 (71%) |
| LN Class II and V | 6 (60%) |
| LN class III and V | 2 (20%) |
| LN Class IV and V | 1 (10%) |
| | 1 (10%) |
| SELENA-SLEDAI at time of urine collection | 4 ± 4 |
| Patients with active LN at time of urine collection | 4 (29%) |
| Renal SLEDAI of the patients with active LN at | 6 ± 2 |
| time of urine collection | |
| Treatment at time of urine collection | |
| Hydroxychloroquine | 10 (71%) |
| Prednisone | 7 (50%) |
| Mycophenolate mofetil | 10 (71%) |
| Azathioprine | 0 (0%) |
| Cyclophosphamide | 0 (0%) |
| Rituximab | 0 (0%) |
| Methotrexate | 1 (7%) |

### MiRNAs in Mesangial Cells

Human renal mesangial cells (ScienCell) were selected for in vitro studies, since the proliferation of this type of cell is a LN-characteristic phenomenon. Mesangial cells were transduced with lentiviruses (Applied Biological Materials: Mh35378, Mh35422, Mh35785; Table 2), at a multiplicity of infection of 1, and in the presence of polybrene (3 μg/mL). The infection efficiency was evaluated by fluorescence with the Axio Observer A1 microscope (Carl Zeiss). Stable knockdowns (KD) were obtained with puromycin selection and were used as polyclonal populations. The controls were mesangial cells transduced with a lentivirus vector and non-infected cells. MiRNA overexpression was achieved by transfection of pCMV-MiR constructs for miR-26a and miR-30b (Origene). These cells were maintained in 20% calf serum.

RNA was extracted with Qiagen RNAeasy kit and analyzed with 2100 Bioanalyzer (Agilent). The transcriptome was amplified with Ovation Pico WTA System V2 (Nugen). Whole genome expression of the KDs and controls was studied using Affymetrix GeneChip Human Gene 2.0 ST arrays. Gene expression data were normalized and quality controls were assessed before further analyses. Canonical pathways were studied using Ingenuity Pathway Analysis (IPA).

The Clontech Advantage RT kit was used to generate cDNA for the study of transcripts. Gene expression was detected by qRT-PCR and normalized to 18S. Commercially available primers were purchased from Applied Biosystems (Table 2).

Cell proliferation was analyzed using the MTT (3-[4,5-dimethylthiazol-2-yl]-2,5-diphenyltetrazolium bromide) assay. The cells were seeded at a density of 100,000 cells/well, cultured for three days and treated according to the manufacturer's protocol. The number of viable cells was calculated according to a standard curve. As a validation strategy, cell proliferation was also measured after six days of culture, using propidium iodide staining (Sigma; 10 mg/ml) with Igepal permeabilization followed by spectrophotometric detection.

### Effect of an Anti-HER2 Drug in Human Mesangial Cells

Mesangial cells were exposed to trastuzumab (8 μg/mL). After 24 h of culture miR-26 and miR-30b were quantified by qRT-PCR, using cel-miR238 and U6 as controls. After three days of culture, RNA was extracted and whole genome expression arrays were performed. The results were compared to mock-treated mesangial cells.

### Immunohistochemistry for HER2 in Humans

Fully automated immunohistochemistry was performed on a Leica Bond-Max, with rabbit anti-HER2 (1:500; 1 hour staining; Sigma-Aldrich), after antigen retrieval at low-pH for 20 min. The Bond polymer refine detection system was used. Images were obtained using Aperio ScanScope eSlide capture device. HER2-positive breast cancer tissue was used as a positive control. For every sample, negative staining controls were executed without the primary antibody.

### Immunohistochemistry for HER2 in a LN Mouse Model

HER2 expression was studied in NZM2410 mice (14), a LN mouse model, and Balb/c and C57BL/6 (B6) mice, non-autoimmune controls, originally obtained from the Jackson Laboratories. Blood and urine were collected every other week from NZM2410 mice. The characteristics of the mice are provided in Table 4. Immunohistochemistry was performed on formalin-fixed kidney tissue with rabbit anti-HER2 (1:300; 1 hour staining; Sigma), after heat antigen retrieval with a low-pH citrate solution (Vector Labs). Amplification with Vectastain avidin/biotinylated enzyme complex and Vector biotinylated anti-rabbit antibody was performed. Negative controls, without primary antibody, were performed in parallel. The number of HER2-positive cells in the glomeruli of NZM2410 mice was compared to controls. On average, 113 glomeruli were analyzed per mouse. A blood urea nitrogen (BUN) higher than 30 mg/dL was considered elevated. All the experiments were approved by the Institutional Animal Care and Use Committee of the Children's Hospital of Philadelphia.

TABLE 4
Characteristics of the mice studied

| Features | NZM2410 | Balb/c | B6 |
|---|---|---|---|
| Total Number | 9 | 3 | 10 |
| Sex | | | |
| Female | 4 (44%) | 3 (100%) | 6 (60%) |
| Male | 5 (56%) | 0 (0%) | 4 (40%) |
| Age at Sacrifice | 6 ± 3 | 8 ± 1 | 4 ± 4 |
| (months) | | | |

### Expression Studies In Vitro

Mesangial cells were treated with α-interferon (Biomedical Laboratories) for three days (100 U/mL) and HER2 expression was evaluated by qRT-PCR and compared to mock-treated cells (primers are listed in Table 2).

Interferon Regulatory Factor 1 (IRF1)-puromycin plasmids and vector-only plasmids were transfected into mesangial cells, using the Amaxa Cell Line Nucleofector Kit V (Lonza). IRF1 transfection was confirmed by qRT-PCR and also by immunofluorescence, using as primary antibody mouse anti-human IRF1 (1:50; 1 h incubation; Santa Cruz) and as secondary antibody Alexa Fluor 546 goat anti-mouse IgG (Invitrogen). The corrected total cell fluorescence was calculated using Image J (NIH). HER2 expression was evaluated by qRT-PCR. MiR-26a and miR-30b were also measured by qRT-PCR, using spiked Caenorhabditis elegans miRNA-39 as control (Qiagen).

### Quantification of HER2, MCP-1 and VCAM-1 in the Urine

Enzyme-linked immunosorbent assays (ELISA) for HER2 (Abcam), monocyte chemotactic protein-1 (MCP-1) (R&D Systems) and vascular cell adhesion molecule 1 (VCAM-1) (R&D Systems) were performed, according to the manufacturers' instructions, using human urine samples. Spectrophotometry was performed with EL808 (Biotek).

Urine samples from LN patients were obtained from the Johns Hopkins Lupus cohort (15). Samples from patients with active lupus nephritis at time of urine collection (renal SLEDAI ≧4) were compared with samples from adult, healthy, gender and age-matched individuals. The characteristics of the patients are available in Table 5. Samples from patients with active LN were also compared with samples from the same patients collected at least three months before the identified flare, when the renal SLEDAI was zero. To assess the association between HER2 and biopsy results, we studied samples that were collected from patients who had a kidney biopsy within a year from the date of the urine collection. The IRB offices of the Children's Hospital of Philadelphia and of the Johns Hopkins University School of Medicine approved this study. Urine samples were collected into sterile containers and a protease inhibitor was added (cOmplete Protease Inhibitor Cocktail, Roche). The samples were placed on ice or refrigerated at 4° C. within 1 h of collection and stored at −80° C. HER2, MCP-1 and VCAM-1 values were normalized by dividing by the urine creatinine concentration.

TABLE 5
Demographic and Clinical Information regarding the cohort
used to study HER2 in the urine

| | Number (%) |
|---|---|
| Characteristics | or Mean ± S.D. |
| Gender | |
| Female | 40 (85%) |
| Male | 7 (15%) |
| Ethnicity | |
| African-American | 28 (60%) |
| Caucasian | 12 (26%) |
| Asian | 3 (6%) |
| Other | 4 (9%) |
| Education (years) | 14 ± 3 |
| Family History of SLE | 15 (32%) |
| Age at SLE diagnosis (years) | 23 ± 10 |
| Age at LN diagnosis (years) | 26 ± 11 |
| Age at time of urine collection (years) | 36 ± 10 |
| Time between LN diagnosis and urine collection | 10 ± 8 |
| (years) | |
| Clinical manifestations of SLE | |
| Malar Rash | 25 (53%) |
| Discoid Rash | 15 (32%) |
| Photosensitivity | 15 (32%) |
| Oral or nasal ulcers | 12 (26%) |
| Arthritis | 32 (68%) |
| Pleuritis | 20 (43%) |
| Pericarditis | 14 (30%) |
| Renal Disorder | 47 (100%) |
| Neurologic Disorder | 15 (32%) |
| (Seizures; psychosis; organic brain syndrome; | |
| cognitive impairment; lupus headache; aseptic | |
| meningitis; stroke; mononeuritis multiplex; | |
| optic neuritis; cranial neuropathy; peripheral | |
| neuropathy and/or transverse myelitis) | |
| Hematologic Disorder | 34 (72%) |
| Hemolytic anemia | 9 (19%) |
| Leukopenia | 25 (53%) |
| Lymphocytopenia | 20 (43%) |
| Thrombocytopenia | 10 (21%) |
| Immunologic Disorder | 45 (96%) |
| Alopecia due to SLE | 20 (43%) |
| Raynaud's phenomenon | 18 (38%) |
| Cutaneous vasculitis | 5 (11%) |
| Positive anti-nuclear antibody | 45 (96%) |
| Antibody Profile | |
| Anti-DNA | 40 (85%) |
| Anti-Sm | 24 (51%) |
| Anti-RNP | 21 (45%) |
| Anti-SSA | 25 (54%)* |
| Anti-SSB | 11 (24%)* |
| Anti-phospholipids antibodies | 31 (66%) |
| SELENA-SLEDAI at time of urine collection | 8 ± 4 |
| Renal SELENA-SLEDAI at time of urine | 6 ± 3 |
| collection | |
| Patients with active LN at time of urine | 47 (100%) |
| collection | |
| Kidney Biopsy performed in a consecutive year | 19 (40%) |
| from the urine collection | |
| LN class III | 9 (47%) |
| LN class IV | 6 (32%) |
| LN class V | 4 (21%) |
| Treatment at time of urine collection | |
| Hydroxychloroquine | 40 (85%) |
| Prednisone | 33 (70%) |
| Mycophenolate mofetil | 31 (66%) |
| Azathioprine | 1 (2%) |
| Cyclophosphamide | 2 (4%) |
| Rituximab | 0 (0%) |
| Methotrexate | 0 (0%) |
| Tacrolimus | 4 (9%) |

*One patient was not tested.

### Statistical Analysis

GraphPad Prism 5.0. was used for the statistical analysis. Unpaired t tests and Mann-Whitney U tests were used for comparisons between samples with normal and non-normal distributions, respectively. The relationship between HER2 and the other urinary biomarkers was defined by Pearson's correlation and linear regression. P values <0.05 were considered statistically significant. In vitro experiments were performed at least three times.

The following example is provided to illustrate certain embodiments of the invention. It is not intended to limit the invention in any way.

### Example I

### The Role of MicroRNAs and Human Epidermal Growth Factor Receptor 2 in Proliferative Lupus Nephritis

Non-invasive strategies are needed for both LN diagnosis and monitoring. Furthermore, treatment of LN (3) is associated with several side-effects, including major infections (4) and infertility (5). The identification of new biomarkers to guide the judicious use of these therapeutic agents and the development of new treatment strategies with fewer side effects would have an enormous impact in the management of LN patients.

Cellular microRNAs (miRNAs) are non-coding RNAs with a key role in the post-transcriptional regulation of gene expression. Each miRNA can regulate hundreds of target mRNAs and thereby control almost every biological pathway (6). MiRNA dysregulation can elicit a dramatic change in cell behavior, contributing therefore to the pathogenesis of human diseases. The development of high-throughput methodologies for the global measurement of miRNAs, and the stability of these molecules in biologic fluids, has allowed them to emerge as a new class of biomarkers.

Several studies of miRNAs in systemic lupus erythematosus (SLE) have been performed in plasma (7), peripheral blood mononucleated cells (8-10) and in the kidneys of LN patients (11,12), however, no unbiased analysis of renal tissues has been performed. We found decreased levels of three miRNAs. Known roles for these miRNAs led us to evaluate HER2 as a biomarker and cell cycle genes as downstream targets. Our findings suggest a model whereby type I interferons upregulate HER2 expression, which downregulates miR-26a and miR-30b, releasing cell cycle transcripts from repression and contributing to the mesangial cell proliferation seen in LN. These data identify a new pathway for therapeutics and also identify a robust biomarker.

### Results

### Lupus Nephritis has a Characteristic Kidney miRNA Signature that Reflects Cell Proliferation

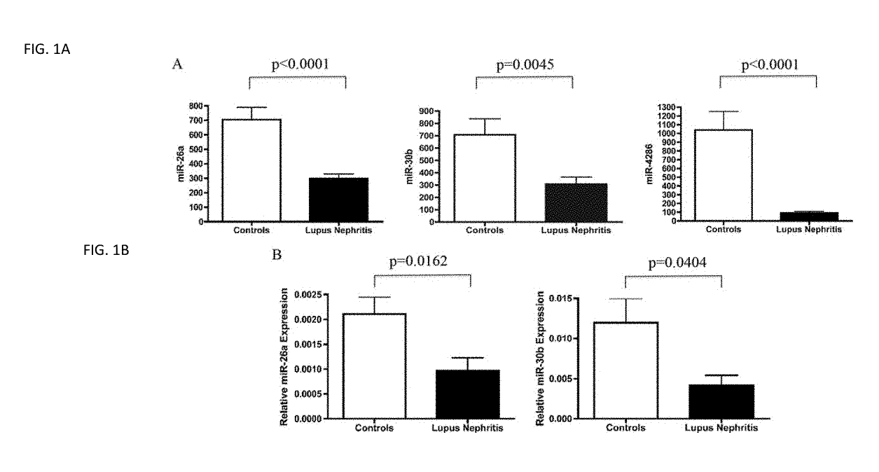

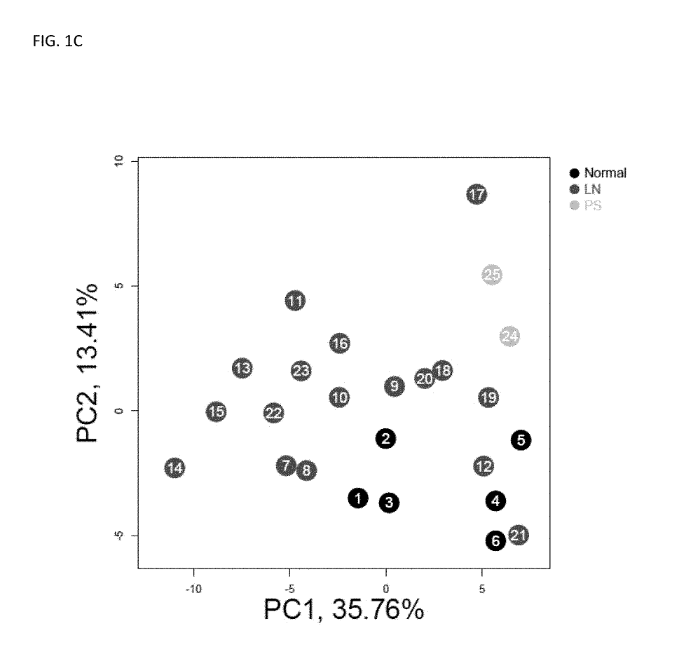

The expression of more than 700 miRNAs was analyzed in the kidneys of controls and children with LN and post-streptococcal glomerulonephritis. Principal component analysis showed that the three groups clustered in different parts of the diagram (FIG. 1C). No differences were seen between the different types of controls. These data indicate that the LN miRNA pattern is characteristic of the disease and reflect its specific pathogenesis. Forty-one miRNAs were significantly decreased in the kidneys of LN patients when compared to controls (p<0.05; Table 6). According to IPA, these miRNAs were associated with cell cycle (p=9.02×10⁻⁰⁵−2.26×10⁻⁰²). We selected three miRNAs for further study: miR-26a, miR-30b and miR-4286 (FIG. 1A), based on the magnitude of the differential expression and p values (p<0.0001; p=0.0045 and p<0.0001, respectively). There were no significant differences in the expression of these miRNAs, according to gender, ethnicity, age or immunosuppressive regimen used (data not shown). Patients without previous treatment also had a statistically significant decrease in miR-26a, miR-30b and miR-4286 when compared to controls (p=0.0005; p=0.0244 and p=0.0003, respectively). Housekeeping genes were no different between LN patients and controls. The data for miR-26a and miR-30b levels were validated by qRT-PCR. The fragmented RNA from the paraffin embedded samples could not be interrogated to confirm a transcriptome signature related to dysregulation of miR-26a and miR-30b, however, we performed an in silico analysis of data available publicly (16). The transcriptome in LN samples showed a significant de-repression of predicted miR-26a and miR30-b targets. DAVID terms enriched in the miR-26a targets upregulated in LN glomeruli were blood pressure, transmembrane proteins, defense response, response to wounding, immune response and lipoproteins (p<10⁻⁴). For miR-30b, the terms were immune response, disulfide bond, regulation of blood pressure, and regulation of proliferation (p<10⁻⁴). This supports the concept that this miRNA pattern is biologically relevant in LN. We next focused our functional analyses on miR-26a and miR-30b, since miR-4286 was recently described and its cellular functions are still unknown.

TABLE 6
miRNAs significantly decreased or increased in the kidneys of patients
with lupus nephritis when compared to controls (p < 0.05).
MiRNAs decreased in the kidneys of patients with LN
when compared to normal kidneys

| | T test Normal | Fold difference |
|---|---|---|
| Gene Name | vs LN | LN/Normal |
| hsa-miR-4286 | 2.12853E−05 | 0.089536526 |
| hsa-miR-26a | 4.36969E−05 | 0.423574285 |
| hsa-miR-23b | 0.000177406 | 0.633675638 |
| hsa-miR-30c | 0.000222236 | 0.363989142 |
| hsa-let-7e | 0.000737622 | 0.661720546 |
| hsa-miR-663a | 0.00359616 | 0.426168113 |
| hsa-miR-1973 | 0.003630868 | 0.339647702 |
| hsa-miR-30b | 0.004535288 | 0.434049392 |
| hsa-let-7a | 0.004561134 | 0.618014422 |
| hsa-miR-145 | 0.00495462 | 0.582368133 |
| hsa-miR-423-5p | 0.005203153 | 0.592909576 |
| hsa-let-7b | 0.00584016 | 0.685095507 |
| hsa-miR-637 | 0.006001927 | 0.619592875 |
| hsa-miR-30e | 0.007082057 | 0.640052785 |
| hsa-miR-626 | 0.009620165 | 0.554575524 |
| hsa-let-7c | 0.013890988 | 0.649720169 |
| hsa-miR-604 | 0.013976949 | 0.675510401 |
| hsa-miR-876-3p | 0.01427286 | 0.572573562 |
| hsa-miR-596 | 0.014412144 | 0.540657199 |
| hsa-miR-339-5p | 0.015042612 | 0.634662327 |
| hsa-let-7f | 0.015236723 | 0.47854804 |
| hsa-miR-29c | 0.01829084 | 0.689901074 |
| hsa-miR-630 | 0.018796748 | 0.221340836 |
| hsa-miR-30a | 0.019323611 | 0.713582478 |
| hsa-miR-20a + hsa-miR-20b | 0.022759215 | 0.66281194 |
| hsa-miR-98 | 0.024476095 | 0.709887433 |
| hsa-miR-4284 | 0.024531451 | 0.567944133 |
| hsa-miR-572 | 0.026212829 | 0.667400419 |
| hsa-miR-22 | 0.030640897 | 0.698466309 |
| hsa-miR-210 | 0.031714249 | 0.450475436 |
| hsa-miR-525-5p | 0.033482294 | 0.687291952 |
| hsa-miR-1298 | 0.037455011 | 0.581829496 |
| hsa-miR-1269a | 0.043284428 | 0.675144988 |
| hsa-miR-578 | 0.043965959 | 0.77804043 |
| hsa-miR-517c-3p + hsa-miR-519a-3p | 0.046464016 | 0.678564482 |
| hsa-miR-422a | 0.048339795 | 0.770921214 |
| hsa-miR-193b-3p | 0.048588633 | 0.614699517 |
| hsa-miR-221 | 0.048655838 | 0.551131207 |
| hsa-miR-1243 | 0.049758824 | 0.710537105 |

MiRNAs increased in the kidneys of patients with LN when
compared to normal kidneys

| | T test Normal | Fold |
|---|---|---|
| | vs LN | difference |
| hsa-miR-577 | 0.002966376 | 1.742928975 |
| hsa-miR-378h | 0.003441046 | 1.679714197 |
| hsa-miR-1273e | 0.003543853 | 1.775357056 |
| hsa-miR-1827 | 0.003841936 | 2.119547757 |
| hsa-miR-548ae | 0.013253079 | 1.748176957 |
| hsa-miR-576-5p | 0.015078496 | 1.87795421 |
| hsa-miR-548j | 0.019630894 | 2.124509804 |
| hsa-miR-758 | 0.019903329 | 1.601457399 |
| hsa-miR-888-5p | 0.023249248 | 1.831257881 |
| hsa-miR-548ac | 0.026315585 | 2.536134088 |
| hsa-miR-921 | 0.027744692 | 1.932698413 |
| hsa-miR-548d-3p | 0.030733223 | 2.659273151 |
| hsa-miR-603 | 0.03107462 | 2.165185916 |
| hsa-miR-570-3p | 0.032523902 | 1.42959854 |
| hsa-miR-1273a | 0.035392959 | 1.639341741 |
| hsa-miR-548b-5p | 0.039407225 | 1.874268191 |
| hsa-miR-154-5p | 0.04336021 | 1.8154 |
| hsa-miR-574-5p | 0.044504049 | 3.471392823 |
| hsa-miR-302d-3p | 0.044835518 | 1.463923036 |
| hsa-miR-548z | 0.045544296 | 2.282964986 |
| hsa-miR-1264 | 0.046666603 | 1.601000196 |

MiR-26a and miR-30b are Decreased in the Urine of LN Patients

We analyzed the urinary levels of miR-26a and miR-30b to explore their potential role as LN biomarkers. We also observed decreased levels of these miRNAs in the urine of adults with LN when compared to healthy controls, just as was the case in renal tissues (p=0.0162; p=0.0404, respectively; FIG. 1B).

Mesangial Cells with miRNA Knockdowns have a Higher Expression of Genes Related to Cell Cycle and DNA Replication

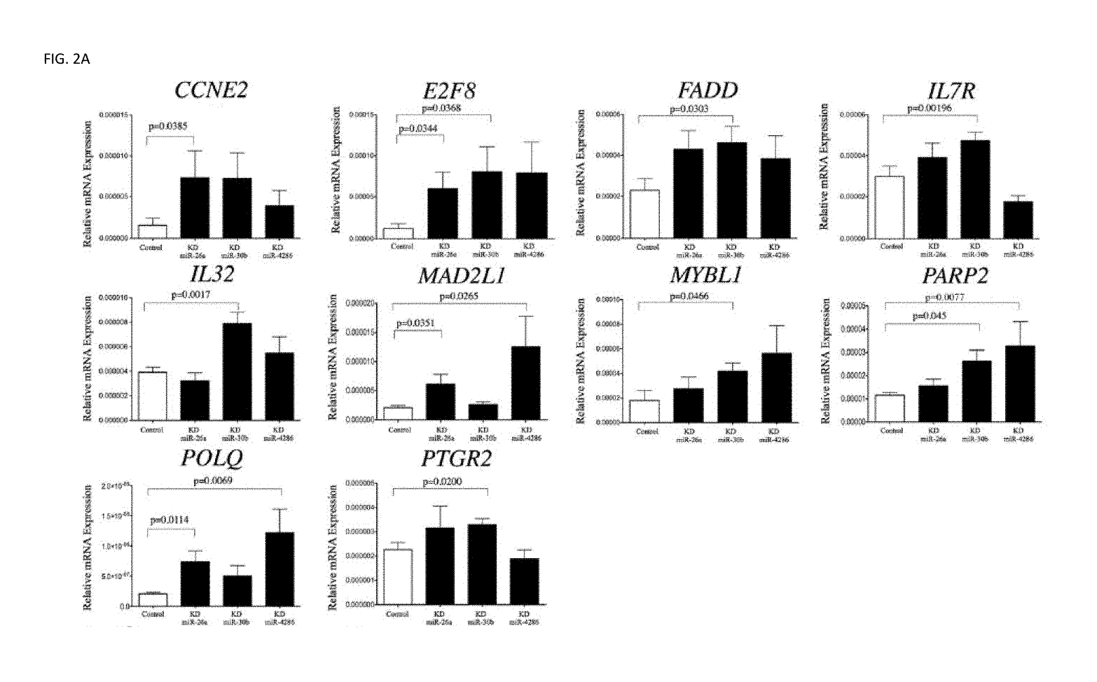

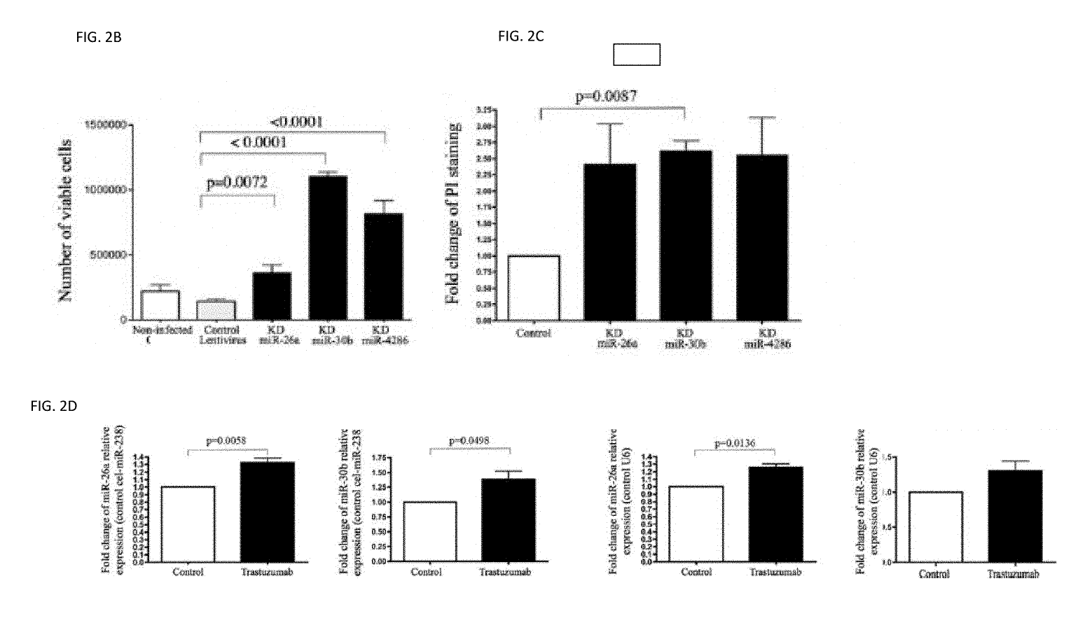

Cell cycle pathways were strongly enriched in our analysis of the LN miRNA signature and proliferation is a hallmark of LN. We therefore investigated whether these miRNAs directly regulated cell cycle gene transcript abundance. We knocked down (KD) each implicated miRNA in human mesangial cells and analyzed the gene expression initially by arrays. According to IPA, the KD of miR-26a led to increased expression of genes associated with cell cycle (p=9.98×10⁻⁹ to 1.98×10⁻²) and DNA replication, recombination and repair (1.10×10⁻⁸ to 1.98×10⁻²). Similar results were obtained for miR-30b KD (1.47×10⁻²⁰ to 1.11×10⁻²; for both types of genes) and miR-4286 KD (9.39×10⁻²² to 1.15×10⁻²; 9.39×10⁻²² to 1.15×10⁻², respectively). These findings suggested that miR-26a, miR-30b and miR-4286 were directly regulating the expression of genes involved in proliferation. The expression levels of 10 genes were validated by qRT-PCR (FIG. 2A). Conversely, when miR-26a and miR-30b overexpression vectors were transfected into mesangial cells, we saw decreased expression of the cell cycle genes. Thus, the miRNA signature appears to be intimately involved in pathologic cell cycle gene expression in LN.

Mesangial Cells with miRNA KD Proliferate More than Those Infected with Lentivirus Controls

We directly examined proliferation by performing MTT assays. A statistically significant increase in the number of viable mesangial cells with miR-26a, miR-30b or miR-4286 KDs was seen, when compared with the cells transduced with the lentivirus control (p=0.0072; p<0.0001; p<0.0001, respectively. FIG. 2B). These data were further confirmed using propidium iodide to measure DNA content (FIG. 2B).

Mesangial Cells Treated with Trastuzumab have a Lower Expression of Genes Associated with the Cell Cycle and have Higher Levels of miR-26a and miR-30b

Trastuzumab, a monoclonal antibody inhibiting HER2 (Human Epidermal Growth Factor Receptor 2), causes a G1 arrest in breast cancer cells by increasing miR-26a and miR-30b (17). We hypothesized that trastuzumab might also affect the cell cycle of mesangial cells. We performed a microarray study on trastuzumab-treated cells. The cells that were exposed to trastuzumab had decreased expression of genes related to cell cycle (p=3.96×10⁻⁵ to 2.70×10⁻²) and DNA replication, recombination and repair (p=1.39×10⁻⁸ to 2.70×10⁻²), as one would expect with inhibition of a growth factor pathway. The same pathways as were seen in the miRNA KD cells were involved, but the effect on gene expression was the opposite. Furthermore, miR-26a and miR-30b levels were measured in trastuzumab-treated cells and found to be increased, using either cel-miR238 or U6 as control (FIG. 2D). These data confirm that in mesangial cells, similar to breast cancer cells, trastuzumab can inhibit cell cycle gene expression and increase miR-26a and miR-30b.

### HER2 is Dramatically Increased in Lupus Nephritis, but not in Other Proliferative Glomerulonephritides

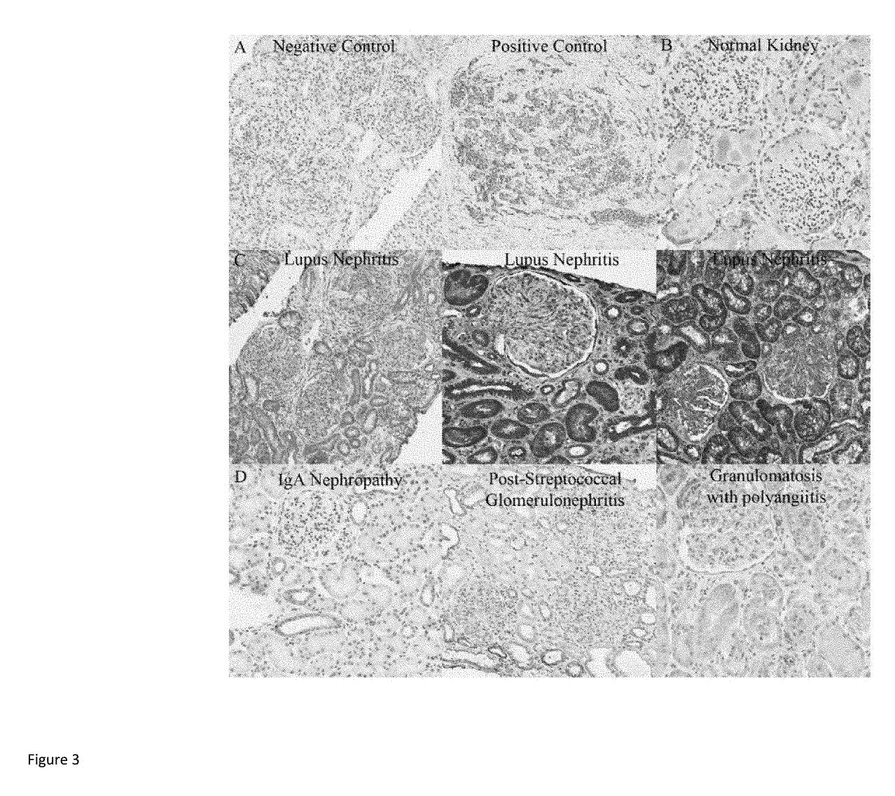

Prior studies have suggested that the HER2 pathway directly regulates the expression of miR-26a and miR-30b. The effects of trastuzumab led us to hypothesize that increased HER2 might be regulating miRNA expression specifically in LN. No prior studies of HER2 expression had been performed in this disease. We found a dramatic increase in HER2 expression in the kidneys of LN patients, not only in the tubular compartment, but also in the glomeruli, where mesangial cells, endothelial cells and podocytes were strongly stained. Normal kidneys and disease controls (IgA nephropathy, post-streptococcal glomerulonephritides and granulomatosis with polyangiitis) had light staining of tubules, but no strongly HER2-positive cells in the glomerulus (FIG. 3).

### HER2 in the Glomeruli of NZM2410 Mice and in Urine

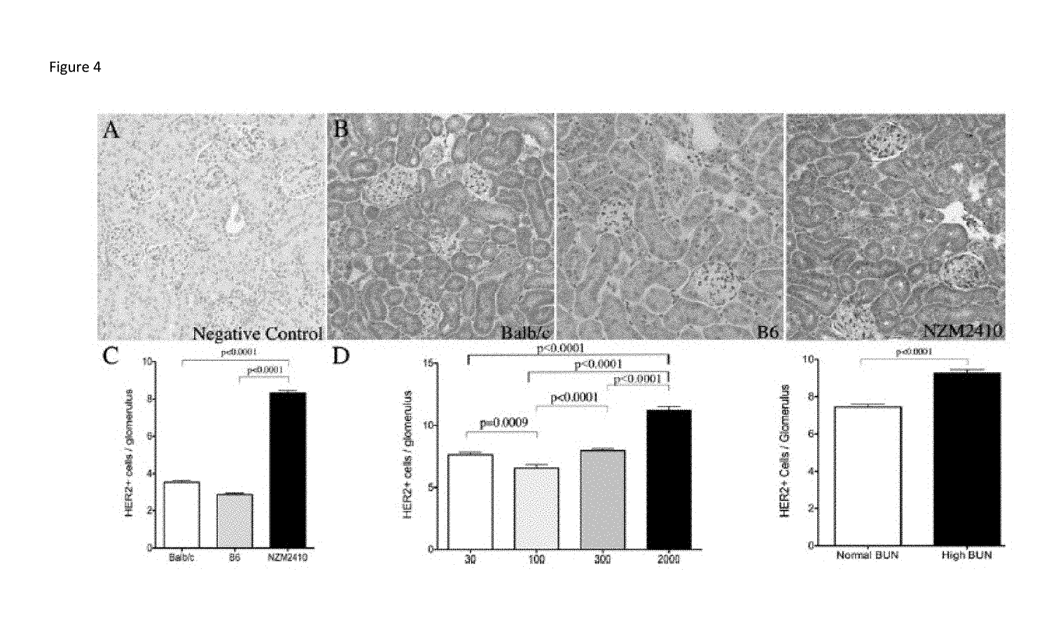

To further examine HER2 in LN, immunohistochemistry studies were also conducted in NZM2410 mice and the control B6 and Balb/c mice (FIG. 4). The NZM2410 mice developed, as expected, an early onset, aggressive, lupus-like diffuse proliferative glomerulonephritis. While in healthy humans HER2 staining was almost absent in the kidneys, in healthy B6 and Balb/c mice, the tubules showed strong HER2 staining, with the glomeruli being negative. Kidneys from NZM2410 mice, however, showed a significantly higher number of HER2-positive cells per glomerulus when compared to B6 and Balb/c mice (p<0.0001 in both cases) (FIG. 4C). In addition, the number of HER2-positive cells per glomerulus was significantly higher in NZM2410 mice with higher proteinuria and higher levels of BUN (FIG. 4D), suggesting that HER2 expression correlates with the severity of the disease.

### α-Interferon and IRF1 Increase the Expression of HER2 in Human Mesangial Cells

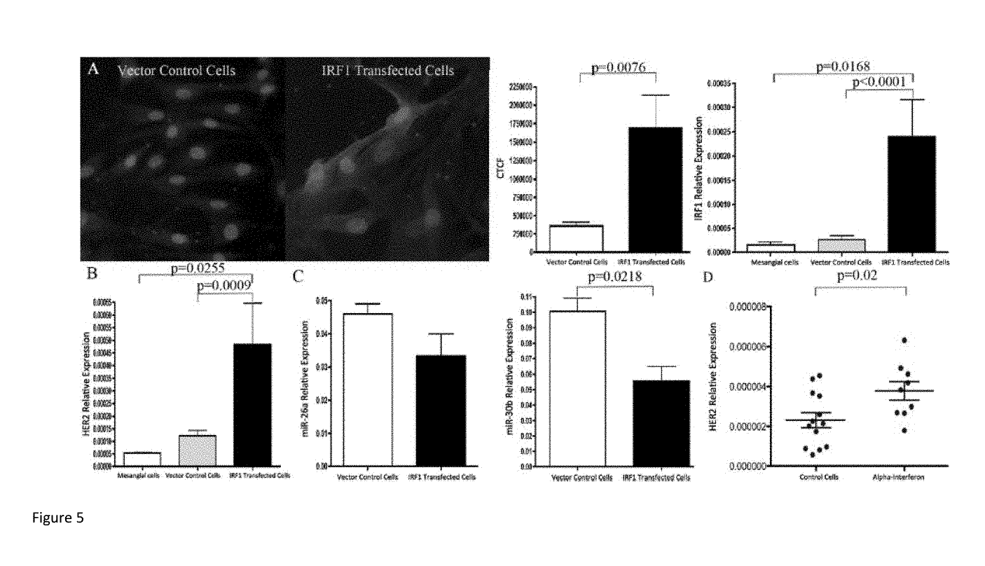

These data implicate a model whereby increased HER2 expression drives decreased miR26a and miR30b, allowing for de-repression of cell cycle transcripts. We next wished to understand the etiology of the increased HER2 expression. Since type I interferons have been implicated in SLE, we analyzed whether α-interferon could regulate HER2 expression. Mesangial cells exposed to α-interferon had a significantly higher expression of HER2 than controls (p=0.02; FIG. 5).

IRF1 is a key transcription factor induced by α-interferon, and we examined its effect on HER2 expression. IRF1 transfection was confirmed by immunofluorescence and qRT-PCR, as shown in FIG. 5. HER2 expression was significantly increased in the IRF1 transfected cells when compared with the cells transfected with the vector (p=0.0009; FIG. 5B). Finally, miR-26a and miR-30b were evaluated and decreased levels were seen in IRF1 transfected cells (FIG. 5C). These data suggested that lupus-associated factors, like α-interferon and IRF1, contribute to the HER2 overexpression and the secondarily decreased miR-26a and miR-30b levels seen in LN.

HER2 is Increased in the Urine of Patients with LN and is Associated with Disease Activity

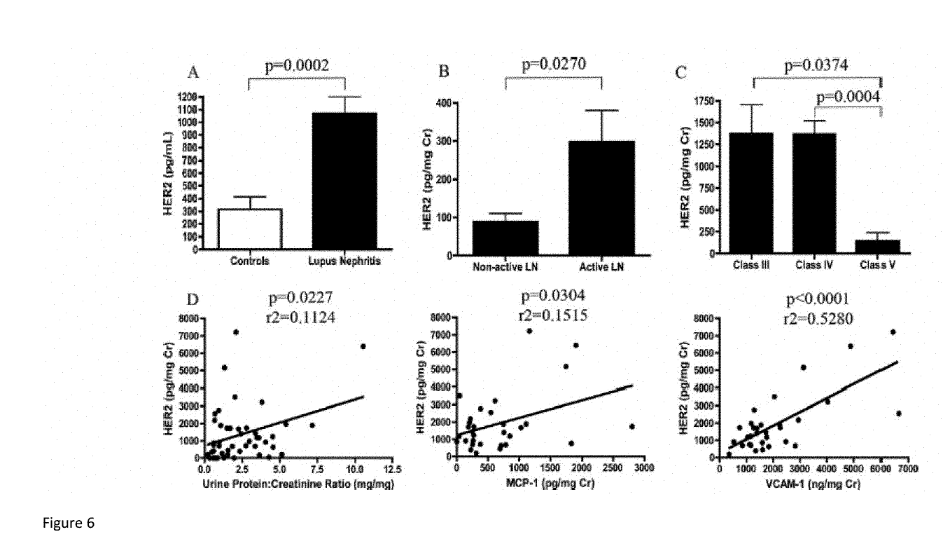

The dramatic overexpression of HER2 in LN renal tissue suggested that it could be a useful biomarker. We, therefore, examined the urine from patients and controls. HER2 was found to be significantly increased in the urine of adult patients with active LN when compared to matched-sex healthy controls (p=0.0002; FIG. 6A). Moreover, when analyzing HER2 levels longitudinally, it was found that they were significantly increased during LN flares (p=0.0270; FIG. 6B). HER2 was also significantly increased in the urine of patients with class III and class IV LN when compared with class V (p=0.0374; p=0.0004, respectively; FIG. 6C). Finally, HER2 levels correlated with the urine protein:creatinine ratio (p=0.0227) and with the levels of other LN biomarkers, namely MCP-1 (p=0.0304) and VCAM-1 (p<0.0001) (FIG. 6D).

MCP-1 and VCAM-1 are thought to direct inflammatory cell infiltrates in LN and have been described as potential biomarkers. In LN tubules, VCAM-1 was identified as a miR-30b target with increased expression among genes. We therefore examined expression of MCP-1 and VCAM-1 in miR-26a and miR-30b overexpressing mesangial cells and found decreased expression of both in the miR-26a overexpressing cells and deceased VCAM-1 in the miR-30b overexpressing cells. These data suggest that both proliferation and inflammatory infiltrate are both influenced by these miRNAs.

### Discussion

Our data support a model where type I interferons, via the transcription factor IRF1, induce the expression of HER2. This pathway down-regulates miR-26a and miR-30b, thereby driving cell proliferation through de-repression of genes involved in the cell cycle.

Our initial discovery study began with pediatric SLE patients. The pediatric age group was selected because LN is particularly prevalent and severe in children with SLE (2,18) and co-morbidities, such as diabetes mellitus or hypertension, are not as frequent. We found a miRNA signature specific for LN: a significant decrease in miR-26a, miR-30b and miR-4286. Manipulation of these miRNAs in human mesangial cells showed that they participate in the cell cycle regulation by controlling the expression of several key genes, including CCNE2, which is involved in the G1/S transition (19,20). CCNE2 had also been predicted to be a target of both miR-26a and miR-30b by the miRanda algorithm.

Decreased miR-26a has also been identified in several malignancies (17,20-38). Consistently, miR-26a overexpression inhibits cell proliferation (24,27,28,39) usually through the control of CCNE2 expression (21,24,39). MiR-26a dysregulation has also been associated with other autoimmune diseases, such as rheumatoid arthritis (40), and idiopathic pulmonary fibrosis (41).

Similarly, miR-30b is associated with cell proliferation (29,42). Down-regulation of miR-26a and miR-30b has also been reported in the sera of patients with scleroderma and systemic sclerosis (43), fibrotic diseases affecting multiple tissues.

In LN, there is a large spectrum of morphological changes in the glomerular, tubulointerstitial and vascular compartment of the kidneys, but cell proliferation is central to its pathogenesis. Not only is mesangial hypercellularity one of the first manifestations of the disease, but the presence of crescents is a well-known negative prognostic factor.

The stability of miRNAs in urine and the ease of obtaining such samples make these molecules suitable candidates as non-invasive biomarkers. We showed that not only are miR-26 and miR-30b significantly decreased in the kidneys of LN, but also in the urine of these patients.

Trastuzumab targets the HER2 extra-cellular domain and blocks its downstream pathways by inhibiting the dimerization of HER2 and by promoting the internalization and cleavage of HER2 molecules. Currently, trastuzumab is used for the treatment of patients with HER2-positive breast and gastric cancer where it causes a G1 arrest by up-regulating miR-26a and miR-30b (17). The mechanism by which HER2 regulates miR26a and miR30b is not known. Our studies showed a dramatic increase of HER2 expression in the kidneys of NZM2410 mice and in LN patients. Furthermore, mesangial cells exposed to trastuzumab had elevated levels of miR-26a and miR-30b and down-regulation of genes associated with mitosis and cell proliferation, demonstrating that this pathway is central to the regulation of miR-26a and miR-30b. Other members of the epidermal growth factor (EGF) family have been shown to participate in the pathogenesis of renal diseases. In a crescentic glomerulonephritis mouse model, the activation of EGF receptor in podocytes resulted in the development or progression of the disease, while the pharmacological blockage or genetic deletion of one of its ligands improved the course of the disease and prevented the infiltration of inflammatory cells (44). Moreover, activation of EGFR in cultured podocytes led to proliferation, dedifferentiation and migration, processes that are thought to occur in crescent formation in vivo (44). We did not find, however, HER2 overexpression in other types of glomerulonephritides also characterized by mesangioproliferation, such as IgA nephropathy, post-streptococcal glomerulonephritis and granulomatosis with polyangiitis. We hypothesized, therefore, that lupus-associated factors drive HER2 overexpression. The effects of α-interferon and IRF1 in human mesangial cells were evaluated, since the role of these two factors in SLE etiopathogenesis has been previously identified (45-47). We showed that both α-interferon and IRF1 increased HER2 expression in human mesangial cells and that IRF1 was also associated with a significant decrease of miR-30b. The ChIP-Seq data available on UCSC Genome Bioinformatics platform are consistent with the binding of IRF1 to the HER2 promoter region in K562 cells, which further supports our observation that IRF1 controls HER2 expression.

HER2 in the urine was significantly increased in LN patients and it was associated with proliferative disease, as expected from its known role in cell growth. Urinary HER2 levels were also significantly correlated with urine protein to creatinine ratio, as well as MCP-1 and VCAM1 levels, recognized LN biomarkers (48-50). HER2 levels likely capture a distinct pathologic mechanism in LN and the association between biomarkers is statistically robust. Our data highlight a novel, previously unexpected pathway and novel biomarkers for LN. For decades, proliferation has been recognized as the hallmark of LN and the identification of this pathway opens the door for novel therapeutic interventions.

### REFERENCES

1. Mok C C, Kwok R C L, Yip P S F. Effect of renal disease on the standardized mortality ratio and life expectancy of patients with systemic lupus erythematosus: SMR and life expectancy of patients with lupus renal disease. Arthritis Rheum 2013; 65:2154-60.
2. Brunner H I, Gladman D D, Ibanez D, Urowitz M D, Silverman E D. Difference in disease features between childhood-onset and adult-onset systemic lupus erythematosus. Arthritis Rheum 2008; 58:556-62.
3. Bertsias G K, Tektonidou M, Amoura Z, Aringer M, Bajema I, Berden J H M, et al. Joint European League Against Rheumatism and European Renal Association-European Dialysis and Transplant Association (EULAR/ERA-EDTA) recommendations for the management of adult and paediatric lupus nephritis. Ann Rheum Dis 2012; 71:1771-82.
4. Costa-Reis P, Nativ S, Isgro J, Rodrigues T, Yildirim-Toruner C, Starr A, et al. Major infections in a cohort of 120 patients with juvenile-onset systemic lupus erythematosus. Clin Immunol 2013; 149:442-9.
5. Harward L E, Mitchell K, Pieper C, Copland S, Criscione-Schreiber L G, Clowse M E B. The impact of cyclophosphamide on menstruation and pregnancy in women with rheumatologic disease. Lupus 2013; 22:81-6.
6. Krek A, Grun D, Poy M N, Wolf R, Rosenberg L, Epstein E J, et al. Combinatorial microRNA target predictions. Nat Genet 2005; 37:495-500.
7. Carlsen A L, Schetter A J, Nielsen C T, Lood C, Knudsen S, Voss A, et al. Circulating microRNA expression profiles associated with systemic lupus erythematosus. Arthritis Rheum 2013; 65:1324-34.
8. Te J L, Dozmorov I M, Guthridge J M, Nguyen K L, Cavett J W, Kelly J A, et al. Identification of unique microRNA signature associated with lupus nephritis. PloS One 2010; 5:e10344.
9. Liu D, Zhao H, Zhao S, Wang X. MicroRNA expression profiles of peripheral blood mononuclear cells in patients with systemic lupus erythematosus. Acta Histochem. 2014; 20.
10. Zhu J, Huang X, Su G, Wang L, Wu F, Zhang T, et al. High expression levels of microRNA-629, microRNA-525-5p and microRNA-516a-3p in paediatric systemic lupus erythematosus. Clin Rheumatol 2014; 33:807-15.
11. Dai Y, Sui W, Lan H, Yan Q, Huang H, Huang Y. Comprehensive analysis of microRNA expression patterns in renal biopsies of lupus nephritis patients. Rheumatol Int 2009; 29:749-54.
12. Lu J, Kwan B C-H, Lai F M-M, Tam L-S, Li E K-M, Chow K-M, et al. Glomerular and tubulointerstitial miR-638, miR-198 and miR-146a expression in lupus nephritis. Nephrol 2012; 17:346-51.
13. Weening J J, D'Agati V D, Schwartz M M, Seshan S V, Alpers C E, Appel G B, et al. The classification of glomerulonephritis in systemic lupus erythematosus revisited. J Am Soc Nephrol 2004; 15:241-50.
14. Colonna L, Dinnall J-A, Shivers D K, Frisoni L, Caricchio R, Gallucci S. Abnormal costimulatory phenotype and function of dendritic cells before and after the onset of severe murine lupus. Arthritis Res Ther 2006; 8:R49.
15. Fangtham M, Petri M. 2013 Update: Hopkins Lupus Cohort. Curr Rheumatol Rep [Internet]. 2013 [cited 2014 Jun. 16]; 15. Available from: http://www.ncbi.nlm.nih.gov/pmc/articles/PMC3756858/16.
16. Berthier C C, Bethunaickan R, Gonzalez-Rivera T, Nair V, Ramanujam M, Zhang W, et al. Cross-species transcriptional network analysis defines shared inflammatory responses in murine and human lupus nephritis. J Immunol 2012; 189:988-1001.
17. Ichikawa T, Sato F, Terasawa K, Tsuchiya S, Toi M, Tsujimoto G, et al. Trastuzumab produces therapeutic actions by upregulating miR-26a and miR-30b in breast cancer cells. PloS One 2012; 7:e31422.
18. Mina R, Brunner H I. Pediatric lupus-are there differences in presentation, genetics, response to therapy, and damage accrual compared with adult lupus? Rheum Dis Clin North Am 2010; 36:53-80, vii-viii.
19. Payton M, Scully S, Chung G, Coats S. Deregulation of cyclin E2 expression and associated kinase activity in primary breast tumors. Oncogene 2002; 21:8529-34.
20. Deng Q, Wang Q, Zong W-Y, Zheng D-L, Wen Y-X, Wang K-S, et al. E2F8 contributes to human hepatocellular carcinoma via regulating cell proliferation. Cancer Res 2010; 70:782-91.
21. Lu J, He M-L, Wang L, Chen Y, Liu X, Dong Q, et al. MiR-26a inhibits cell growth and tumorigenesis of nasopharyngeal carcinoma through repression of EZH2. Cancer Res 2011; 71:225-33.
22. Jia L-F, Wei S-B, Gan Y-H, Guo Y, Gong K, Mitchelson K, et al. Expression, regulation and roles of miR-26a and MEG3 in tongue squamous cell carcinoma. Int J Cancer 2013.
23. Zhang Y-F, Zhang A-R, Zhang B-C, Rao Z-G, Gao J-F, Lv M-H, et al. MiR-26a regulates cell cycle and anoikis of human esophageal adenocarcinoma cells through Rbl-E2F1 signaling pathway. Mol Biol Rep 2013; 40:1711-20.
24. Kota J, Chivukula R R, O'Donnell K A, Wentzel E A, Montgomery C L, Hwang H-W, et al. Therapeutic microRNA delivery suppresses tumorigenesis in a murine liver cancer model. Cell 2009; 137:1005-17.
25. Ji J, Shi J, Budhu A, Yu Z, Forgues M, Roessler S, et al. MicroRNA expression, survival, and response to interferon in liver cancer. N Engl J Med 2009; 361:1437-47.
26. Yang X, Liang L, Zhang X-F, Jia H-L, Qin Y, Zhu X-C, et al. MicroRNA-26a suppresses tumor growth and metastasis of human hepatocellular carcinoma by targeting interleukin-6-Stat3 pathway. Hepatol 2013; 58:158-70.
27. Zhang B, Liu X-X, He J-R, Zhou C-X, Guo M, He M, et al. Pathologically decreased miR-26a antagonizes apoptosis and facilitates carcinogenesis by targeting MTDH and EZH2 in breast cancer. Carcinogenesis 2011; 32:2-9.
28. Gao J, Li L, Wu M, Liu M, Xie X, Guo J, et al. MiR-26a inhibits proliferation and migration of breast cancer through repression of MCL-1. PloS One 2013; 8:e65138.
29. Gao W, Shen H, Liu L, Xu J, Xu J, Shu Y. MiR-21 overexpression in human primary squamous cell lung carcinoma is associated with poor patient prognosis. J Cancer Res Clin Oncol 2011; 137:557-66.
30. Dang X, Ma A, Yang L, Hu H, Zhu B, Shang D, et al. MicroRNA-26a regulates tumorigenic properties of EZH2 in human lung carcinoma cells. Cancer Genet 2012; 205:113-23.
31. Visone R, Pallante P, Vecchione A, Cirombella R, Ferracin M, Ferraro A, et al. Specific microRNAs are downregulated in human thyroid anaplastic carcinomas. Oncogene 2007; 26:7590-5.
32. Lv M, Zhang X, Li M, Chen Q, Ye M, Liang W, et al. miR-26a and its target CKS2 modulate cell growth and tumorigenesis of papillary thyroid carcinoma. PloS One 2013; 8:e67591.
33. Heinzelmann J, Henning B, Sanjmyatav J, Posorski N, Steiner T, Wunderlich H, et al. Specific miRNA signatures are associated with metastasis and poor prognosis in clear cell renal cell carcinoma. World J Urol 2011; 29:367-73.
34. Wang G, Zhang H, He H, Tong W, Wang B, Liao G, et al. Up-regulation of microRNA in bladder tumor tissue is not common. Int Urol Nephrol 2010; 42:95-102.
35. Ciarapica R, Russo G, Verginelli F, Raimondi L, Donfrancesco A, Rota R, et al. Deregulated expression of miR-26a and Ezh2 in rhabdomyosarcoma. Cell Cycle 2009; 8:172-5.
36. Song Q-C, Shi Z-B, Zhang Y-T, Ji L, Wang K-Z, Duan D-P, et al. Downregulation of microRNA-26a is associated with metastatic potential and the poor prognosis of osteosarcoma patients. Oncol Rep 2014; 31:1263-70.
37. Kozubek J, Ma Z, Fleming E, Duggan T, Wu R, Shin D-G, et al. In-depth characterization of microRNA transcriptome in melanoma. PloS One 2013; 8:e72699.
38. Sand M, Skrygan M, Georgas D, Sand D, Hahn S A, Gambichler T, et al. Microarray analysis of microRNA expression in cutaneous squamous cell carcinoma. J Dermatol 2012; 68:119-26.
39. Deng J, He M, Chen L, Chen C, Zheng J, Cai Z. The loss of miR-26a-mediated post-transcriptional regulation of cyclin E2 in pancreatic cancer cell proliferation and decreased patient survival. PloS One 2013; 8:e76450.
40. Murata K, Furu M, Yoshitomi H, Ishikawa M, Shibuya H, Hashimoto M, et al. Comprehensive microRNA analysis identifies miR-24 and miR-125a-5p as plasma biomarkers for rheumatoid arthritis. PloS One 2013; 8:e69118.
41. Liang H, Gu Y, Li T, Zhang Y, Huangfu L, Hu M, et al. Integrated analyses identify the involvement of microRNA-26a in epithelial-mesenchymal transition during idiopathic pulmonary fibrosis. Cell Death Dis 2014; 5:e1238.
42. Liao W-T, Ye Y-P, Zhang N-J, Li T-T, Wang S-Y, Cui Y-M, et al. MicroRNA-30b functions as a tumour suppressor in human colorectal cancer by targeting KRAS, PIK3CD and BCL2: miR-30b target genes in CRC. J Pathol 2014; 232:415-27.
43. Tanaka S, Suto A, Ikeda K, Sanayama Y, Nakagomi D, Iwamoto T, et al. Alteration of circulating miRNAs in SSc: miR-30b regulates the expression of PDGF receptor β. Rheumatol 2013; 52:1963-72.
44. Bollée G, Flamant M, Schordan S, Fligny C, Rumpel E, Milon M, et al. Epidermal growth factor receptor promotes glomerular injury and renal failure in rapidly progressive crescentic glomerulonephritis. Nat Med 2011; 17:1242-50.
45. Pascual V, Farkas L, Banchereau J. Systemic lupus erythematosus: all roads lead to type I interferons. Curr Opin Immunol 2006; 18:676-82.
46. Costa-Reis P, Sullivan K E. Genetics and epigenetics of systemic lupus erythematosus. Curr Rheumatol Rep 2013; 15:369.
47. Zhang Z, Song L, Maurer K, Petri M A, Sullivan K E. Global H4 acetylation analysis by ChIP-chip in systemic lupus erythematosus monocytes. Genes Immun 2010; 11:124-33.
48. Watson L, Midgley A, Pilkington C, Tullus K, Marks S, Holt R, et al. Urinary monocyte chemoattractant protein 1 and alpha 1 acid glycoprotein as biomarkers of renal disease activity in juvenile-onset systemic lupus erythematosus. Lupus 2012; 21:496-501.
49. Wu T, Xie C, Wang H W, Zhou X J, Schwartz N, Calixto S, et al. Elevated urinary VCAM-1, P-selectin, soluble TNF receptor-1, and CXC chemokine ligand 16 in multiple murine lupus strains and human lupus nephritis. J Immunol 2007; 179:7166-75.
50. Watson L, Tullus K, Pilkington C, Chesters C, Marks S D, Newland P, et al. Urine biomarkers for monitoring juvenile lupus nephritis: a prospective longitudinal study. Pediatr Nephrol 2014; 29:397-405.

While certain preferred embodiments of the present invention have been described and specifically exemplified above, it is not intended that the invention be limited to such embodiments. Various modifications may be made to the invention without departing from the scope and spirit thereof as set forth in the following claims.

What is claimed is:

1. A method of detecting HER2 in the urine of a patient at risk for, or having Lupus nephritis (LN), comprising the steps of:
a) contacting a urine sample obtained from said patient with an antibody having binding affinity for HER2 or a nucleic acid which hybridizes to a HER2 encoding nucleic acid;
b) detecting formation of a specific binding pair comprising an antibody-HER2 immunocomplex or a duplex of a HER2 specific probe or primer and a HER2 encoding nucleic acid present in said urine sample; said detection of said specific binding pair indicating an increased risk for, or the presence of LN.

2. The method of claim 1, comprising detection of miR-26a and miR-30b levels in said sample.

3. The method of claim 2, comprising detection of VCAM1 and MCP-1 in said sample.

4. The method of claim 1, wherein HER2 protein in urine is detected using an anti-HER2 antibody or a functional fragment thereof, said method comprising;
i) contacting said urine sample with said anti-HER2 antibody, under conditions that an immunocomplex forms between HER2 and said anti-HER2 antibody;
(ii) washing the immunocomplex to remove unbound HER2 protein;
(iii) adding a detectably labeled antibody so as to allow formation of a complex between said detectably labeled antibody and said anti-HER2 antibody bound HER2 protein; and
(iv) detecting said complex, wherein the presence of said complex is indicative of the presence of HER2 in said urine, indicative of an increased risk for, or the presence of LN in said human.

5. The method of claim 1, further comprising assessing said patient for additional LN symptoms selected from peripheral edema secondary to hypertension, headache, dizziness, visual disturbances, cardiac decompensation, hypoalbuminemia, fatigue, fever, rash, arthritis, serositis and central nervous system disease.

6. The method of claim 2, further comprising performing a renal biopsy on said patient to classify the type of LN present in said patient.

7. The method of claim 1, further comprising treating said subject with an agent effective alleviate symptoms of LN.

8. The method of claim 7, wherein said agent is selected from one or more of hydroxychloroquine, glucocorticoids, cyclophosphamide, mycophenolate mofetil, angiotensin converting enzyme inhibitors, angiotensin receptor blockers, prednisone, and immunosuppressive agents.

9. The method of claim 1, wherein the HER2 protein is detected by an immunological assay selected from the group consisting of enzyme linked immunosorbent assay (ELISA), Western blotting, immunoprecipitation assay, immunochromatography, radioimmunoassay (RIA), Radioimmunodiffusion, immunofluorescence assay (IFA), immunoblotting, Ouchterlony immunodiffusion, rocket immunoelectrophoresis, immunohistostaining, complement fixation assay, fluorescence activated cell sorting (FACS) and protein chip assay.

10. The HER2 detecting method of claim 4, wherein the level of HER2-antibody immunocomplex formation is measured by quantitatively analyzing color development with a secondary antibody conjugated with a label selected from the group consisting of an enzyme, a fluorescent, a luminescent, a microparticle, a redox molecule, and a radioisotope, said secondary antibody being able to bind to the second antibody.

11. The method of claim 10 comprising quantifying miR-26a and miR-30b in said sample via qRTPCR.

12. The method of claim 10, comprising quantifying VCAM1 and MCP-1 in said sample.

13. A kit for detecting lupus nephritis in a human in the method of claim 1, comprising:
(a) antibodies immunologically specific for HER2, VCAM-1 and MCP-1; and
(b) instructional materials for the use of said monoclonal antibody in detecting HER2, and optionally VCAM-1 and MCP-1 protein;
c) isolated HER2 protein for use as a control.

14. The kit of claim 13, further comprising a microtiter plate or a dipstick, said microtiter plate or dipstick having HER2 antibody or HER2 protein immobilized thereon.

15. The kit of claim 13, further comprising a detection reagent and a receptacle for collection of said urine sample.

16. The kit of claim 15, wherein said detection reagent is a secondary antibody conjugated with a label selected from the group consisting of an enzyme, a fluorescent, a luminescent, a microparticle, a redox molecule, and a radioisotope, said secondary antibody being able to bind to the HER2 specific antibody.

17. The kit of claim 13, further comprising reagents suitable for quantifying miR-26a and miR-30b using qRT-PCR.

18. The kit of claim 13, comprising probes or primers of sufficient complementarity to bind HER2 encoding nucleic acids, said probes or primers optionally being labeled with a non-naturally occurring detectable label.

19. The method of claim 1, wherein said patient is experiencing a LN flare.

20. The method of claim 1, wherein said patient has Class III or Class IV LN.

21. The method of claim 7, wherein said agent is trastuzumab.

22. The method of claim 7, wherein said agent is herceptin.
